(19) United States
(12) Patent Application Publication
(10) Pub. No.: US 2019/0104933 A1
(43) Pub. Date: Apr. 11, 2019

(54) DEVICES AND METHOD FOR FAR FIELD BIPOLAR ABLATION

(71) Applicant: NewUro, B.V., Amsterdam (NL)

(72) Inventors: Roger A. STERN, Cupertino, CA (US); Jerome JACKSON, Los Altos, CA (US); Benjamin WANG, San Leandro, CA (US); Omry BEN-EZRA, Tel Aviv (IL); Itzhak AVNERI, Tel Aviv (IL); Lior AVNERI, New York, NY (US)

(21) Appl. No.: 16/210,990

(22) Filed: Dec. 5, 2018

Related U.S. Application Data
(63) Continuation of application No. PCT/US17/36212.
(60) Provisional application No. 62/346,095, filed on Jun. 6, 2016.

(51) Int. Cl. A61B 1/307; A61B 18/14; A61N 1/32

(52) U.S. Cl. CPC A61B 1/307; A61B 2018/0016; A61N 1/32; A61B 18/1492

(57) ABSTRACT

The present disclosure describes devices and methods for treating disorders in a hollow body organ by ablating the tissue therein. At least one set of bipolar electrodes is deployed in the hollow body organ to contact the inner wall of the organ. In the deployed position, each positive electrode is positioned in a location substantially opposite each negative electrode. The tissue contact areas of the positive and negative electrodes are substantially the same and the electrodes are separated from one another by a distance of at least 10 times the width of each of the electrodes. The electrodes thereby produce lesions that are substantially identical to one another and also similar to those produced with monopolar electrodes. The electrodes are used to produce an ablation pattern that can electrically isolate regions of the hollow body organ, thereby treating the disorder(s).

### CROSS-REFERENCE

This application is a continuation of PCT Application No. PCT/US17/36212, filed Jun. 6, 2017, which claims the benefit of U.S. Provisional Application No. 62/346,095, filed Jun. 6, 2016, which applications are incorporated herein by reference in their entirety.

The subject matter of this application is related to that of the following co-pending patent applications: PCT Application No. PCT/IB2016/000953, filed Jun. 10, 2016, and U.S. patent application Ser. No. 15/179,623, filed Jun. 10, 2016 both of which claim priority to U.S. Provisional Patent Application No. 62/174,296, filed Jun. 11, 2015, which applications are incorporated herein by reference.

The subject matter of this application is also related to that of the following co-pending patent applications: PCT Application No. PCT/IB2014/003083, filed Nov. 25, 2014, which claims the benefit of U.S. patent application Ser. No. 14/519,933, filed Oct. 21, 2014, which is a continuation-in-part application of PCT Application Serial No. PCT/IB2013/001203, filed Apr. 19, 2013, which claims the benefit of U.S. Provisional Application Nos. 61/636,686, filed Apr. 22, 2012, and 61/649,334, filed May 20, 2012, and PCT Application No. PCT/IB2014/003083 also claims the benefit of U.S. Provisional Applications Nos. 61/908,748, filed Nov. 26, 2013, and 61/972,441, filed Mar. 31, 2014, which applications are incorporated herein by reference; and U.S. patent application Ser. No. 14/602,493, filed Jan. 22, 2015, which is a continuation of U.S. patent application Ser. No. 14/519,933, which applications are incorporated herein by reference.

### BACKGROUND

The current disclosure relates to systems, devices, and methods for medical treatment, particular by the ablation of tissue such as with radiofrequency (RF) energy.

RF ablation within body organs has been extensively described previously and is well known in the art. Where elongated or extensive lesions with large surface areas are desired, the two main technologies utilizing RF energy are monopolar ablation and bipolar ablation.

In monopolar ablation, a treating electrode is placed where a lesion is created, and the current flows through the tissues to a dispersive electrode. The dispersive electrode has a large surface area compared to the treating electrode, so that current density over this dispersive electrode is low enough to prevent any lesion from forming. This dispersive, or “patient”, or “ground” electrode is usually placed on the patient's skin in a location such as the thighs or flanks.

Examples of internal organ ablation applications employing monopolar ablation include pulmonary vein isolation for atrial fibrillation and ablation of tumors in soft tissue such as in the liver.

Problems with this technology include situations in which contact between the dispersive electrode and the skin is suboptimal, in which case, current density increases and a skin lesion might form, in some cases associated with serious burns and injury. Worse yet, complete disconnection of the ground electrode might cause current to flow through a different route, which might endanger vital organs. Another disadvantage of monopolar ablation is the tendency of lesions to be more pronounced at the edge of the electrode (“edge effect”). This effect can be minimized by using relatively short electrodes, however this necessitates more wires.

In bipolar ablation, both electrodes are considered “treating” electrodes and are usually approximately identical in dimensions and electrical properties and placed very close to each other, usually less than 1 cm apart. The current flows between the two electrodes through the tissues; and, as the electrodes are identical and proximate, the tissues between them are more or less homogenously ablated. In this configuration, in order to achieve a linear lesion, two adjacent electrodes are needed, with two separate wires (“railway” like configuration).

Examples of internal organ ablation applications employing bipolar ablation include esophageal ablation with the Barrx device available from Medtronic PLC of Dublin, Ireland.

An advantage of this technology is the ability to control lesion depth with high accuracy. Problems with this technology are mainly associated with the limited area that can be treated by such electrodes, so that if a large ablation area is desired, a large number of electrodes must be used, resulting with many wires leading to them, which increased the diameter of elements in the device.

There remains a need for a technology that enables ablation within hollow organs in a manner that allows safe, easy, and quick creation of homogenous lesions having large areas, preferably elongated but optionally having a large surface area such as circular or oblong shaped, while using a simple device having few electrical wires running through it, and a low profile of insertion.

### SUMMARY

To solve the need outlined above, the current disclosure describes a technique herein called “Far-Field-Bipolar” ablation, as well as specific device embodiments which may employ this technique.

The far field bipolar technique may utilize bipolar electrodes having substantially equal surface areas, positioned at a relatively large distance from each other within the target organ, such that they may produce lesions similar to those produced with monopolar electrodes.

The device embodiments described in this disclosure may comprise an expandable element which may appose the electrodes to the target organ wall, and typically may be capable of stretching the electrodes to facilitate their collapse and removal from the patient's body.

An aspect of the present disclosure provides devices for treating a disorder in a hollow body organ. An exemplary device may comprise a shaft having a distal tip, at least one set of bipolar electrodes, and an expandable member configured to radially expand the at least one set of bipolar electrodes from a folded or compressed position to a deployed position. Each set of bipolar electrodes may comprise at least one first polarity electrode and at least one second polarity electrode. In the deployed position, each first polarity electrode may be configured to be positioned in a location within the hollow body organ substantially opposite each second polarity electrode. In the deployed position, the distance between each electrode pair may be at least 10 times the width of each of the electrodes.

A total tissue contact area of the at least one first polarity electrode of each set of bipolar electrodes may be substantially equal to a total surface area of the at least one second polarity electrode of the same set of bipolar electrodes.

The device may be configured for creating a predetermined pattern of electrically isolated tissue regions having reduced electrical propagation in an inner wall of the hollow body organ such that electrical propagation through the hollow body organ as a whole is reduced. Each electrode may comprise an elongate conductor.

The at least one set of bipolar electrodes may comprise four sets of bipolar electrodes. Two sets of bipolar electrodes may be longitudinal electrode sets which are configured to be substantially parallel to the longitudinal axis of the shaft in the deployed position. Two sets of bipolar electrodes may be two circumferential electrode sets which are configured to be substantially transverse to the longitudinal axis of the shaft in the deployed position. The predetermined pattern of electrically isolated tissue regions having reduced electrical propagation may comprise eight longitudinal splines and a circumferential line. Each set of longitudinal electrodes may comprise four distal electrode segments arranged in a “cross” pattern around the distal tip of the shaft, and four proximal electrode segments may be arranged to be positioned equidistantly between the four distal electrode segments and at a more proximal position. Each set of circumferential electrodes may comprise a first pair of circumferential electrode segments arranged opposite each other in a circumferential line around the expandable element in the deployed position, and a second pair of circumferential electrode segments may be arranged opposite each other in the gaps between the first pair of circumferential electrodes. Each set of longitudinal electrodes may comprise four distal electrode segments arranged in a “flattened x” pattern around the distal tip of the shaft, and four proximal electrode segments arranged such that two of the four proximal electrode segments may be positioned between the four distal electrode segments and at a more proximal position, and each set of circumferential electrodes may comprise a first pair of circumferential electrode segments arranged adjacent each other in a circumferential line around the expandable element in the deployed position and a second pair of circumferential electrode segments may be arranged adjacent each other opposite said first pair of circumferential electrode segments in a circumferential line around the expandable element in the deployed position.

The electrodes may comprise a flexible printed circuit material. The longitudinal electrodes may comprise a flexible printed circuit material, and the circumferential electrodes may comprise a wire or braid. All first polarity electrode segments of each set and all second polarity electrode segments of each set may be connected to each other but not to any other electrode segments via a printed circuit board located at the distal tip of the shaft. The tissue contact area of each electrode set is between 1 mm² and 50 mm².

The device may further comprise one or more wires configured to deliver power to a PCB pass via the shaft.

The device may further comprise an atraumatic cap at the distal tip of the shaft.

The expandable member may comprise a balloon or bladder.

The distance between the electrode pairs may be at least 10 mm.

The at least one set of bipolar electrodes may be printed on the expandable member.

The expandable member may be made of a non-compliant material or a compliant material.

The electrodes create a pattern that is asymmetrical. The pattern may be configured to spare an area of the hollow organ. The at least one first polarity electrode may comprise at least one positive electrode. The at least one second polarity electrode may comprise at least one negative electrode.

The hollow organ may be any of a urinary bladder, uterus, rectum, large or small bowel, stomach, pulmonary artery, cardiac atrium, cardiac ventricle, and the disorder is any of overactive bladder, Detrusor-sphincter dyssynergia, irritable uterus, menorrhagia, irritable bowel, obesity, asthma, atrial fibrillation, ventricular tachycardia.

The at least one set of bipolar electrodes may comprise a conductive flexible or gelatinous material layer on surfaces thereof.

The expandable member may comprise a plurality of parts welded together in a manner such that the expandable member has no outward protruding seam. The plurality of parts may be comprise flanges that are welded together using one or more of forceps, rollers, or clamps.

The at least one set of bipolar electrodes may be configured to protrude from the expandable member when the expandable member is expanded.

The at least one first polarity electrode and the at least one second polarity electrodes may have different surface areas to localize treatment to one of the at least one first polarity or at least one second polarity electrode.

Another aspect of the present disclosure provides devices for treating a disorder in a hollow body organ. An exemplary device may comprise: a handle having a distal end, a proximal end, and a slot; an inner shaft having a distal tip, a proximal end, a stopper and at least one opening; an outer shaft slideably positioned over the inner shaft and having a distal tip, a proximal end, a seal, and an outer shaft base; an outer sheath slideably positioned over the outer shaft and having a distal end, a proximal end, and a valve; at least one set of electrodes each having a distal end and a proximal end and comprising at least one electrode segment; and a balloon having a distal leg and a proximal leg. The proximal end of the inner shaft may be connected to the handle. The outer shaft base may further comprise a retraction knob which slideably protrudes through the slot of the handle. An inflation tube and wires may enter the handle and may be sealed to the inner shaft. The distal leg of the balloon may be connected to the inner shaft proximate the distal tip of the inner shaft and the proximal leg of the balloon may be connected to the outer shaft proximal the distal tip of the outer shaft. The wires may pass through the inner shaft and out of the distal tip of the shaft and connect to the at least one set of electrodes. The proximal end of the at least one electrodes may be connected as a ring slideably positioned over the outer shaft proximal to the proximal leg of the balloon.

The device may have a folded or compressed position and a deployed position and further comprises an atraumatic cap connected to the inner shaft distal tip. The atraumatic cap may be configured to either partially or completely cover the outer sheath distal end when in the folded or compressed position. The outer sheath may be configured to expose the electrodes when pulled proximally. The balloon may be configured to radially expand the electrodes when inflated. The electrodes may be configured to deliver energy to the hollow organ. The outer shaft may be configured to stretch the balloon and collapse the electrodes when pulled proximally by the retraction knob.

The at least one set of electrodes may comprise longitudinal and circumferential electrodes. The longitudinal electrodes may comprise a flexible printed circuit material. The circumferential electrodes may comprise a flexible printed circuit material. The circumferential electrodes may be foldable. The circumferential electrodes may have at least one joint. The at least one joint may comprises a hinge. The joint may comprise an area of the circumferential electrodes with longitudinal zig-zag cuts. A wire may be used to unfold the circumferential electrodes. A conductor of the circumferential electrodes may be on the back side of a printed circuit board (PCB).

The electrodes may create a pattern that is asymmetrical. The pattern may be configured to spare an area of the hollow organ.

The stopper may be positioned distally on the inner shaft such that when the handle is pushed distally, the balloon is configured to further expand radially and expand the electrodes further radially. The balloon may be made of a compliant material or a non-compliant material.

The hollow organ may be any of a urinary bladder, uterus, rectum, large or small bowel, stomach, pulmonary artery, cardiac atrium, cardiac ventricle, and the disorder is any of overactive bladder, Detrusor-sphincter dyssynergia, irritable uterus, menorrhagia, irritable bowel, obesity, asthma, atrial fibrillation, ventricular tachycardia.

The at least one set of bipolar electrodes may comprise a conductive flexible or gelatinous material layer on surfaces thereof. The at least one set of bipolar electrodes may be configured to protrude from the expandable member when expanded.

Another aspect of the present disclosure provides methods for treating a disorder in a hollow body organ. An expandable member may be positioned in the hollow body organ. The expandable member may be expanded in the hollow body organ to expand at least one set of bipolar electrodes from a folded or compressed position to a deployed position in the hollow body organ. The at least one set of bipolar electrodes may comprise at least one first polarity electrode and at least one second polarity electrode. Each first polarity electrode may be positioned in a location within the hollow body organ substantially opposite each second polarity electrode when the at least one set of bipolar electrodes is the deployed position in the hollow body organ. In the deployed position, the distance between each first polarity electrode and the second polarity electrode opposite of said first polarity electrode may be at least 10 times the width of each of the first polarity and second polarity electrodes.

With the at least one set of bipolar electrodes, a predetermined pattern of electrically isolated tissue regions having reduced electrical propagation may be created in an inner wall of the hollow body organ such that electrical propagation through the hollow body organ as a whole is reduced. The predetermined pattern may comprise at least one longitudinal splice and at least one circumferential line. The predetermined pattern may comprise a “cross” pattern or a “flattened x” pattern. The tissue contact area of each electrode set is between 1 mm² and 50 mm². The distance between the at least one first polarity electrode and the at least one second polarity electrode may be at least 10 mm.

The hollow organ may be any of a urinary bladder, uterus, rectum, large or small bowel, stomach, pulmonary artery, cardiac atrium, cardiac ventricle, and the disorder is any of overactive bladder, Detrusor-sphincter dyssynergia, irritable uterus, menorrhagia, irritable bowel, obesity, asthma, atrial fibrillation, ventricular tachycardia.

The expandable member may comprise a balloon, and the expandable member may be expanded by inflating the balloon.

The at least one set of bipolar electrodes may be energized via at least one longitudinal connector connected to and delivering power to the at least one set of bipolar electrodes.

An atraumatic sheath tip may cover a distal end of the expandable member.

Fluid around the expandable member may be removed after the expandable member is expanded in the hollow body organ.

The expandable member in the hollow body organ may be expanded so that the at least one set of bipolar electrodes conform to an inner surface of the hollow body organ. The at least one set of bipolar electrodes may comprise a conductive flexible or gelatinous material layer on surfaces thereof.

The expandable member may comprise a plurality of parts welded together in a manner such that the expandable member has no outward protruding seam. The plurality of parts may comprise flanges that are welded together using one or more of forceps, rollers, or clamps.

Expanding the expandable member in the hollow body organ may cause the at least one set of bipolar electrodes to protrude from the expandable member.

The at least one first polarity electrode and the at least one second polarity electrodes may have different surface areas to localize treatment to one of the at least one first polarity or at least one second polarity electrode. The at least one first polarity electrode may comprise at least one positive electrode. The at least one first polarity electrode may comprise at least one negative electrode.

Another aspect of the present disclosure provides devices treating a disorder in a hollow body organ. An exemplary device may comprise a shaft having a distal tip, an expandable member disposed on the shaft configured to radially expand within the hollow body organ, and a light source within the expandable member. At least the portion of the expandable member may be translucent or transparent to allow light generated from the light source to project from the expandable member to one or more of illuminate or ablate an inner surface of the hollow body organ.

Another aspect of the present disclosure may provide methods for treating a disorder in a hollow body organ. An expandable member may be positioned in the hollow body organ. The expandable member may be expanded in the hollow body organ. Light may be projected from within the expandable member through at least a portion of the expandable member that is translucent to one or more of illuminate or ablate an inner surface of the hollow body organ.

Various improvements and modifications to the above, intended for improving and monitoring surface contact of the electrodes, automation of procedure stages, as well as various other embodiments, are also described.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1A is a schematic coronal section of a urinary bladder showing an ablation pattern, according to many embodiments.

FIG. 1B is a schematic bottom-up view of a urinary bladder showing an ablation pattern, according to many embodiments.

FIG. 2A is a top view of an electrode structure over a spherical expandable element, according to many embodiments.

FIG. 2B is a side view of an electrode structure over a spherical expandable element, according to many embodiments.

FIG. 2C is a top perspective view of an electrode structure over a spherical expandable element, according to many embodiments.

FIG. 3 is a schematic two dimensional representation of an electrode structure, according to many embodiments.

FIG. 4A is a schematic two dimensional representation of an electrode structure, describing a far-field-bipolar ablation energy coupling combination, according to many embodiments.

FIG. 4B is a schematic two dimensional representation of an electrode structure, describing a far-field-bipolar ablation electrode activation sequence, according to many embodiments.

FIG. 4C is a schematic two dimensional representation of an electrode structure, describing a far-field-bipolar ablation electrode activation sequence, according to many embodiments.

FIG. 4D is a schematic two dimensional representation of an electrode structure, describing a far-field-bipolar ablation electrode activation sequence, according to many embodiments.

FIG. 5A is a schematic two dimensional representation of an electrode structure, describing a far-field-bipolar ablation electrode activation sequence, according to many embodiments.

FIG. 5B is a schematic two dimensional representation of an electrode structure, describing a far-field-bipolar ablation electrode activation sequence, according to many embodiments.

FIG. 5C is a schematic two dimensional representation of an electrode structure, describing a far-field-bipolar ablation electrode activation sequence, according to many embodiments.

FIG. 5D is a schematic two dimensional representation of an electrode structure, describing a far-field-bipolar ablation electrode activation sequence, according to many embodiments.

FIG. 6 is a simplified longitudinal cross-section of a device utilizing a flexible PCB material, according to many embodiments.

FIG. 7A describes the electrode structure and wiring of a device, according to many embodiments.

FIG. 7B is a schematic drawing of an alternative circumferential electrode wiring scheme of a device, according to many embodiments.

FIG. 7C is a schematic three dimensional sketch of circumferential electrode attachment methods of a device, according to many embodiments.

FIG. 8A is a simplified schematic longitudinal section of a device in its folded or compressed state, according to many embodiments.

FIG. 8B is a simplified schematic longitudinal section of a device in its deployed and inflated state, according to many embodiments.

FIG. 8C is a simplified schematic longitudinal section of a device in its folded or compressed state, with a different atraumatic tip, according to many embodiments.

FIG. 8D is a simplified schematic longitudinal section of a device in its deployed and inflated state, with a different atraumatic tip, according to many embodiments.

FIG. 9A is a schematic drawing of a circumferential electrode segment of a device, according to many embodiments.

FIG. 9B is a schematic drawing of a circumferential electrode segment of a device, according to many embodiments.

FIG. 9C is a schematic drawing of a circumferential electrode segment of a device, according to many embodiments.

FIG. 9D is a schematic drawing of a circumferential electrode segment of a device, according to many embodiments.

FIG. 10A is a schematic drawing of a circumferential electrode segment of a device, according to many embodiments.

FIG. 10B is a schematic drawing of a circumferential electrode segment of a device, according to many embodiments.

FIG. 10C is a schematic drawing of a circumferential electrode segment of a device, according to many embodiments.

FIG. 11 is a simplified schematic side view of an asymmetrical electrode structure over a spherical expandable element, according to many embodiments.

FIG. 12 is a simplified schematic longitudinal section of a device enabling application of axial force by pushing a retraction knob, according to many embodiments.

FIG. 13A is a simplified schematic longitudinal section of a device in its deployed and inflated state, showing various fluid removal options, according to many embodiments.

FIG. 13B is a simplified schematic axial cross section of a device in its deployed and inflated state, showing features for fluid removal, according to many embodiments.

FIG. 13C is a simplified schematic axial cross section of a device in its deployed and inflated state, showing features for fluid removal, according to many embodiments.

FIG. 13D is a simplified schematic axial cross section of a device in its deployed and inflated state, showing features for fluid removal, according to many embodiments.

FIG. 14A is a simplified schematic section of an electrode segment of a device, according to many embodiments.

FIG. 14B is a simplified schematic section of an electrode segment of a device with a conductive hydrogel layer, according to many embodiments.

FIG. 14C is a simplified schematic section of an electrode segment of a device with a conductive hydrogel layer and tissue, according to many embodiments.

FIG. 15 is a simplified schematic graph showing inflation volumes and pressures of a device, according to many embodiments.

FIG. 16A is a simplified schematic axial cross section of a welded balloon, according to many embodiments.

FIG. 16B is a simplified schematic axial cross section of a welded balloon, according to many embodiments.

FIG. 16C is a simplified schematic three dimensional sketch of a welded balloon, according to many embodiments.

FIG. 16D is a simplified schematic three dimensional sketch of a tool for forming a welded balloon, according to many embodiments.

FIG. 16E is a simplified schematic three dimensional sketch of a tool for forming a welded balloon, according to many embodiments.

FIG. 16F is a simplified schematic three dimensional sketch of the manufacturing process of a welded balloon, according to many embodiments.

FIG. 16G is a simplified schematic three dimensional sketch of a tool for forming a welded balloon, according to many embodiments.

FIG. 16H is a front view of the manufacturing process of a welded balloon, according to many embodiments.

FIG. 16I is a simplified schematic cross section of a welded balloon, according to many embodiments.

FIG. 16J is a simplified schematic cross section of a welded balloon, according to many embodiments.

FIG. 17A is a simplified schematic three dimensional sketch of needle electrodes, according to many embodiments.

FIGS. 17A-E are simplified schematic longitudinal sections of needle electrodes, according to many embodiments.

FIG. 18 is a simplified schematic three dimensional sketch of a localized treatment device, according to many embodiments.

FIG. 19 is a simplified schematic longitudinal section of a light based ablation device, according to many embodiments.

### DETAILED DESCRIPTION

Far Field Bipolar Technique.

To solve the need outlined above, the current disclosure describes a technique herein called “Far-Field-Bipolar” ablation.

This technique is based on use of bipolar electrodes or electrode sets having substantially equal, and relatively large surface areas, that may be positioned at a large distance from each other, relative to the size of the electrodes, and in an almost opposing location within the treated organ. Thus, current flowing from one set of electrodes to the other, may create identical lesions over both electrode sets, similar to monopolar lesions, while sparing the tissue between the electrodes.

To produce the desired effect, the distance between the electrodes should preferably be at least 10 times the relevant dimension of the electrodes, which in the case of elongate electrodes, may typically be the width of the electrode (or its diameter in case of a wire electrode). In the applications described herein, the distance may typically be 1-10 cm.

Since several electrodes can be connected together to form a set of electrodes, as long as the total surface area of each set of electrodes is no larger than approximately 20 mm². This can allow for a relatively small number of wires to be used, while eliminating the need for a dispersive “patient” electrode, as well as the risks and hassle associated with use of such electrodes. An additional important aspect of this technology may be that total ablation time can be significantly reduced (more lesions are created at the same time). The short ablation time can be significant in increasing the appeal of the treatment (to both doctors and patients) and in reducing the pain or discomfort that might be associated with such treatment under local or regional anesthesia.

While this technology may be compatible with use in any body organ, it will be described herein in the context of the urinary bladder, where it may be used for performing Transurethral Bladder Partitioning (“TBP”) for treatment of overactive bladder or other micturition disorders, for performing bladder auto-augmentation by ablating certain layers of the bladder wall, or for any other therapy necessitating extensive or homogenous ablative treatment within the bladder.

TBP is a treatment wherein a transurethral device is used for creating a predetermined pattern of isolated tissue regions in the urinary bladder, such that electrical propagation through the organ as a whole is reduced, thereby treating any number of urinary disorders.

Specific Electrode Energy Coupling Combinations.

The far field bipolar technique described above, as well as any of the devices and methods in the current disclosure, may be used for creation of various ablation patterns in different body organs.

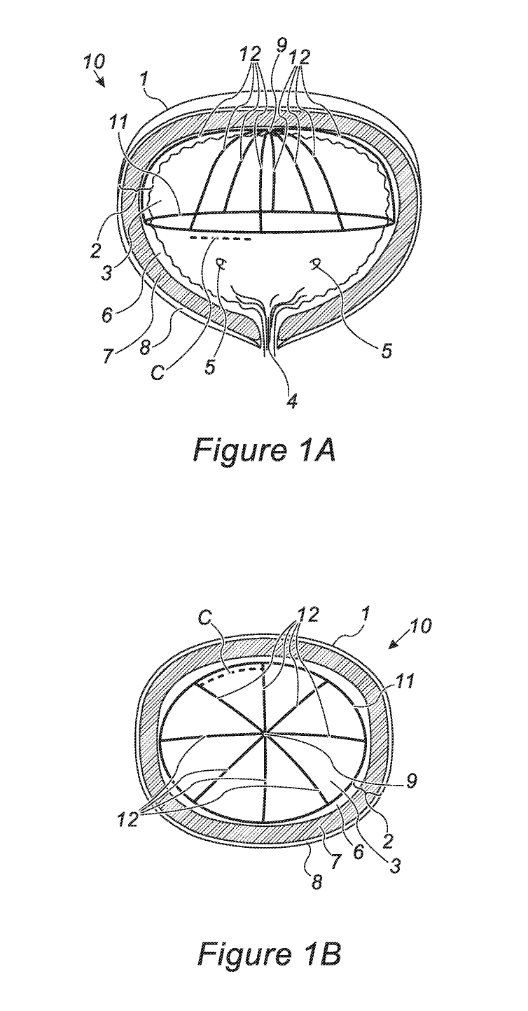

In the current bladder application, a target ablation pattern may be shaped as eight longitudinal splines and a circumferential equatorial line on the upper hemisphere of the bladder as shown in FIGS. 1A and 1B, although any other pattern, such as a hemispherical pattern only, fewer longitudinal lines or a larger number of thereof, a circumferential line only, or other patterns, are within the scope of this disclosure.

FIGS. 1A and 1B describe a target ablation pattern shaped as eight longitudinal splines and a circumferential equatorial line on the upper hemisphere of the bladder.

More particularly, FIG. 1A is a schematic coronal section of a urinary bladder 1 having a bladder wall 2, a bladder lumen 3, a bladder outlet 4, and two ureteral orifices 5. Bladder wall 2 is composed of an inner layer comprising mucosa and submucosa 6, an intermediate layer comprising detrusor muscle 7, and an outer layer comprising adventitia 8. The upper-most point of bladder 1 is its apex 9.

Within bladder 1 is seen a schematic side view of an ablation pattern 10, in this embodiment comprising an equatorial circumferential line 11, and eight longitudinal splines 12 spanning from apex 9 to circumferential line 11, dividing it into 8 equal segments. One such segment is labeled C.

FIG. 1B is a bottom-up view of an axial cross section of urinary bladder 1 showing bladder wall 2, bladder lumen 3, and apex 9 at the center of the figure. Bladder wall 2 is seen composed of mucosa and submucosa 6, detrusor muscle 7, and adventitia 8.

Within bladder 1 is seen a schematic bottom view of ablation pattern 10, comprising circumferential line 11, and eight longitudinal splines 12 spanning from apex 9 to circumferential line 11, dividing it into 8 equal segments. One such segment is labeled C.

This pattern may be configured to limit the free conductance (or communication) of electrical, neural, or other activity between adjacent bladder zones. In particular, this lesion pattern can limit the conduction or communication of excitatory signals traveling in directions other than along the long axis of the bladder. This configuration can be desirable since it may allow the normal conductance associated with micturition (preliminary along the long axis) to occur, while limiting the pathological, disorganized conductance associated with overactive bladder syndromes. For example, a signal originating at a certain point along the bladder wall (above the mid bladder line) may need to traverse 8 lines (all the longitudinal splines) to make a full circle around the bladder perimeter, while crossing only two lines (crossing the circumferential line twice) to make a full circle along the long axis of the bladder.

The ablation patterns described herein are shown on the surface of the bladder; however, their depth can be another important aspect. The depth of ablation may typically include any or all of the layers of the bladder, namely the mucosa and submucosa 6, detrusor 7, and adventitia or serosa 8.

Mucosa 6 typically further comprises an inner-most layer of the mucosa called the urothelium, and the lamina propria, and detrusor 7 typically further comprises an inner longitudinal muscle layer, a middle circumferential muscle layer, and an outer longitudinal muscle layer. Ablation may target any one of the above layers, part of a layer, or a combination of layers.

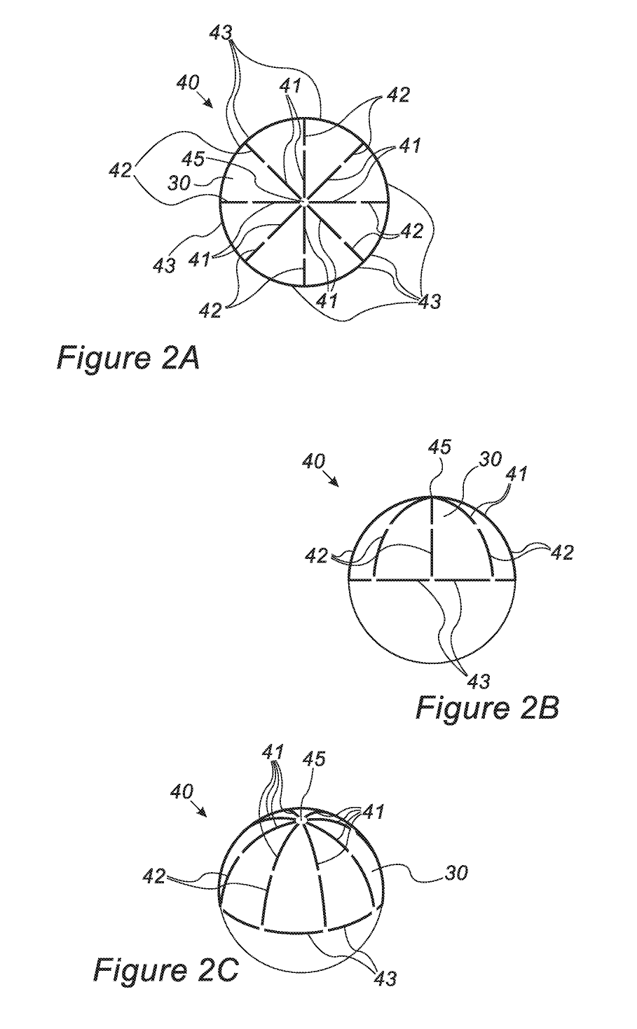

FIGS. 2A-2C are top, side, and three dimensional views of an electrode structure over a spherical expandable element, showing how ablation pattern 10 can be achieved using 24 electrode segments, two segments in each longitudinal spline for a total of 16 longitudinal segments, and a total of eight segments in the whole circumferential line.

More particularly, FIG. 2A is a top view of spherical expandable element 30 in its expanded state, covered by electrode structure 40, having distal longitudinal electrode segments 41 radiating from its center 45, proximal longitudinal electrode segments 42 continuing the lines of segments 41 in a radial direction, and circumferential electrode segments 43, creating a circumferential line around the equator of spherical expandable element 30. Narrow gaps are seen between the ends of each electrode segment and the adjacent segments. This electrode structure may for example be configured to create ablation pattern 10 in a urinary bladder.

FIGS. 2B and 2C are a side view and a perspective three dimensional view respectively, of spherical expandable element 30 in its expanded state, covered by electrode structure 40, having distal longitudinal electrode segments 41 radiating from center 45, seen here at the upper end of spherical expandable element 30, towards its circumference, proximal longitudinal electrode segments 42 continuing the lines of segments 41, and circumferential electrode segments 43, creating a circumferential line around the equator of spherical expandable element 30. Thin gaps are seen between the ends of each electrode segment and the adjacent segments. This electrode structure may be configured to create ablation pattern 10 in a urinary bladder.

In many embodiments, expandable element 30 may be an elastic compliant balloon, or a noncompliant balloon, either referred to herein as a “balloon”. Other spherical expandable elements including cages or similar structures are within the scope of this disclosure.

Of note, a compliant balloon, made for example of silicone, latex, or low durometer polyurethane, may have the advantage of being more easily folded or compressed into a small diameter, as it may be stretched, which reduces its wall thickness.

In contrast, although more difficult to fit into a small diameter sheath, a non-compliant or semi-compliant balloon, for example made of PET, PEBAX, cross linked polyurethane, Nylon, Mylar, polyester, polyurethane, and other polymers in a crosslinked or non-crosslinked form etc., may be advantageous as it may create better wall apposition between the electrodes and organ wall. When inflated to a high pressure, a non-compliant balloon may become rigid, thus preventing bulging of the balloon between the electrodes, or “sinking” of the electrodes into the balloon. Instead, a non-compliant balloon inflated to a rigid state may force the electrodes to slightly bulge out into the target tissue.

In structure 40, all electrode segments 41, 42, and 43 may have substantially the same length, which may be approximately ⅛th of the sphere's circumference. In a human bladder inflated to ˜170 cc, this would typically correspond to a length of ˜27 mm. In some embodiments, the circumferential electrode segments may be longer than the longitudinal electrode segments, allowing the balloon to inflate more than the above mentioned volume. When inflated to a higher volume, the circumferential electrodes may move up the balloon, to encircle the balloon at a higher latitude (above the equator line).

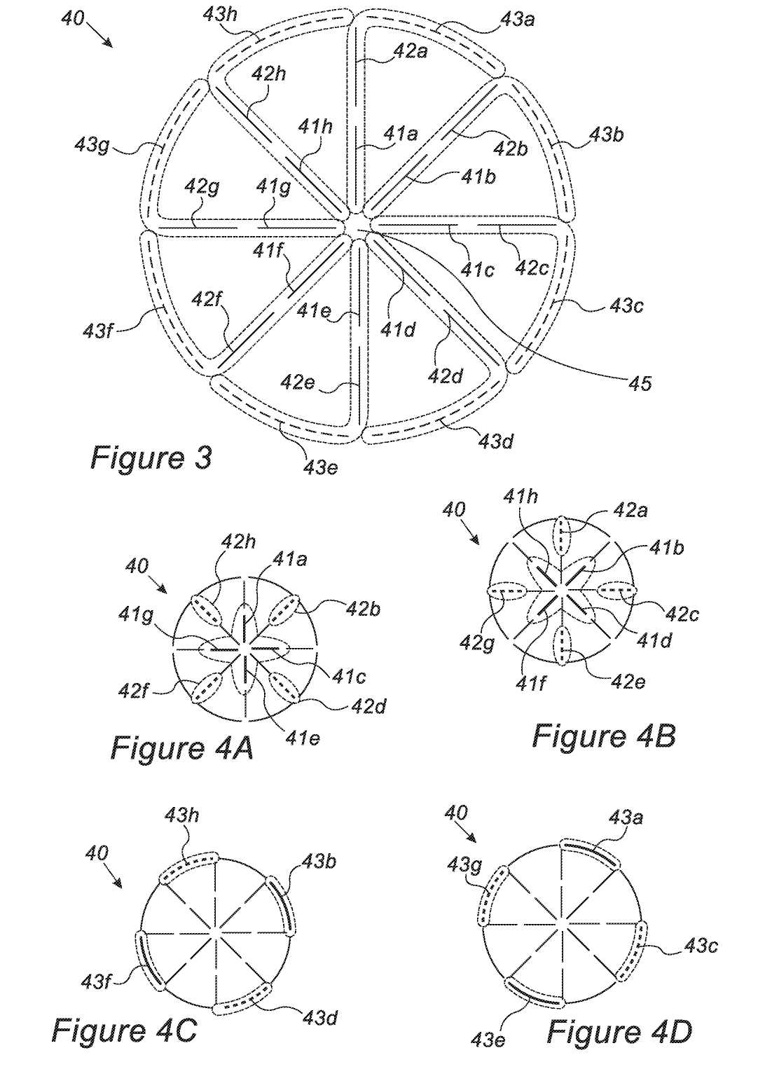

FIG. 3 is a schematic two dimensional representation of electrode structure 40. It should be noted that because FIG. 3 is a two-dimensional projection of a three-dimensional structure, although in the graphic representation different electrode segments seem to have different lengths, in this embodiment all segments may equal an eighth of the circumference of expandable element 30, and therefore may all have the same length.

Shown in FIG. 3 are distal longitudinal electrode segments 41a-h, closer to the apex 45, proximal longitudinal electrode segments 42a-h, closer to the equator line, and circumferential electrode segments 43a-h, which form an equatorial line.

In FIGS. 3-5, electrode segments are labeled with an additional letter “a” through “h” on their reference numbers denoting their location around center 45. For example, electrode segments 41a and 42a are at 12 o'clock, 43a spans the arc between 12 o'clock and 1:30, 41b and 42b are at 1:30, 43b spans the arc between 1:30 and 3 o'clock, and so on. This labeling will be used below when referring to specific segments.

Various combinations of electrode energy coupling and electrode activation sequences can be used with this structure.

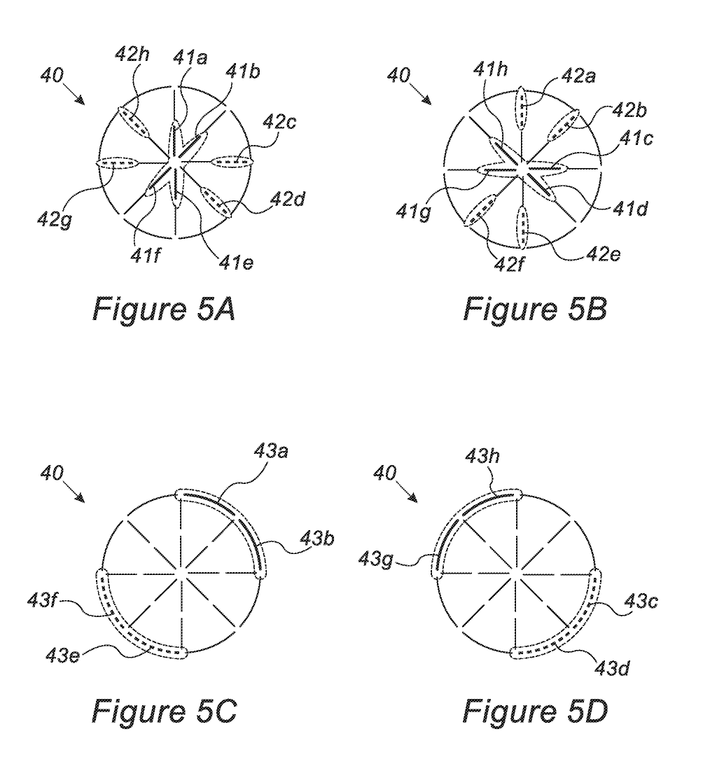

Two such electrode activation sequences employing far field bipolar ablation are shown in FIGS. 4-5. In these figures, the same schematic two-dimensional representation of electrode structure 40 as in FIG. 3 is used, with the electrode set activated at each phase of ablation encircled with a thin dashed line, the group of electrodes acting as one pole marked with a heavy continuous line, and the other group acting as the other pole marked with a heavy dashed line.

Each of FIGS. 4a-4d may represent a single phase of electrode activation during an ablation procedure sequence embodiment. During each such phase of electrode activation, power may be delivered to the active set of electrode segments (shown encircled by a thin dashed line). Transition between the phases may typically be sequential, i.e. after completion of ablation in each phase, the next phase becomes active, so there is a total of four phases. Alternatively, power may be continuously alternated between all phases until ablation of all is complete at about the same time.

More particularly, FIG. 4A shows four equally distributed distal longitudinal electrode segments 41a, 41c, 41e, and 41g, creating a cross pattern around center 45, activated in parallel as one pole, while four proximal longitudinal electrode segments 42b, 42d, 42f, and 42h, distributed equidistantly between the distal electrode segments, may be activated in parallel as the other pole. This may produce half of the longitudinal lines pattern.

FIG. 4B shows the other four equally distributed distal longitudinal electrode segments 41b, 41d, 41f, and 41h, creating a cross pattern around center 45, activated in parallel as one pole, while four proximal longitudinal electrode segments 42a, 42c, 42e, and 42g, distributed equidistantly between the distal electrode segments, may be activated in parallel as the other pole. This may complete the missing half of the longitudinal lines pattern.

FIG. 4C shows two opposing circumferential electrode segments 43b and 43f activated in parallel as one pole, while two opposing circumferential electrode segments 43d and 43h, distributed equidistantly between the first pair of electrode segments, may be activated in parallel as the other pole. This produces half of the circumferential line pattern.

FIG. 4D shows the other two opposing circumferential electrode segments 43a and 43e activated in parallel as one pole, while two opposing circumferential electrode segments 43c and 43g, distributed equidistantly between the first pair of electrode segments, may be activated in parallel as the other pole. This may complete the missing half of circumferential line pattern.

Each of FIGS. 5a-5d may represent a single phase of electrode activation during another ablation procedure sequence embodiment. During each such phase of electrode activation, power may be delivered to the active set of electrode segments (shown encircled by a thin dashed line). Transition between the phases may typically be sequential, i.e., after completion of ablation in each phase, the next phase becomes active, so there is a total of four phases. Alternatively, power may be continuously alternated between all phases until ablation of all is complete at about the same time.

FIG. 5A shows four distal longitudinal electrode segments 41a, 41b, 41e, and 41f, creating a flattened X pattern around center 45, activated in parallel as one pole, while four proximal longitudinal electrode segments 42c, 42d, 42g, and 42h, distributed equidistantly between the distal electrode segments, may be activated in parallel as the other pole. This may produce half of the longitudinal lines pattern.

FIG. 5B shows the other four distal longitudinal electrode segments 41c, 41d, 41g, and 41h, creating a flattened X pattern around center 45, activated in parallel as one pole, while four proximal longitudinal electrode segments 42a, 42b, 42e, and 42f, distributed equidistantly between the distal electrode segments, may be activated in parallel as the other pole. This may complete the missing half of the longitudinal lines pattern.

FIG. 5C shows two adjacent circumferential electrode segments 43a and 43b activated in parallel as one pole, while two opposing adjacent circumferential electrode segments 43e and 43e, may be activated in parallel as the other pole. This produces half of the circumferential line pattern.

FIG. 5D shows another two adjacent circumferential electrode segments 43c and 43d activated in parallel as one pole, while two opposing adjacent circumferential electrode segments 43g and 43h, may be activated in parallel as the other pole. This may complete the missing half of circumferential line pattern.

It is important to note that although in the embodiment described herein pattern 10 is created using 24 electrode segments powered in four separate phases, far-field bipolar may be used to create basically any pattern, using any number of electrode segments and powering phases. As an example, for creating the current ablation pattern 10, using a greater number of segments (for example 48 instead of 24), powered in a larger number of phases (for example 8 instead of 4) is possible. This may be provide the advantage of increasing control and consistency of the lesions created by each individual segment, but this may come in the price of increased complexity of the device, generator, manufacturing, and overall cost.

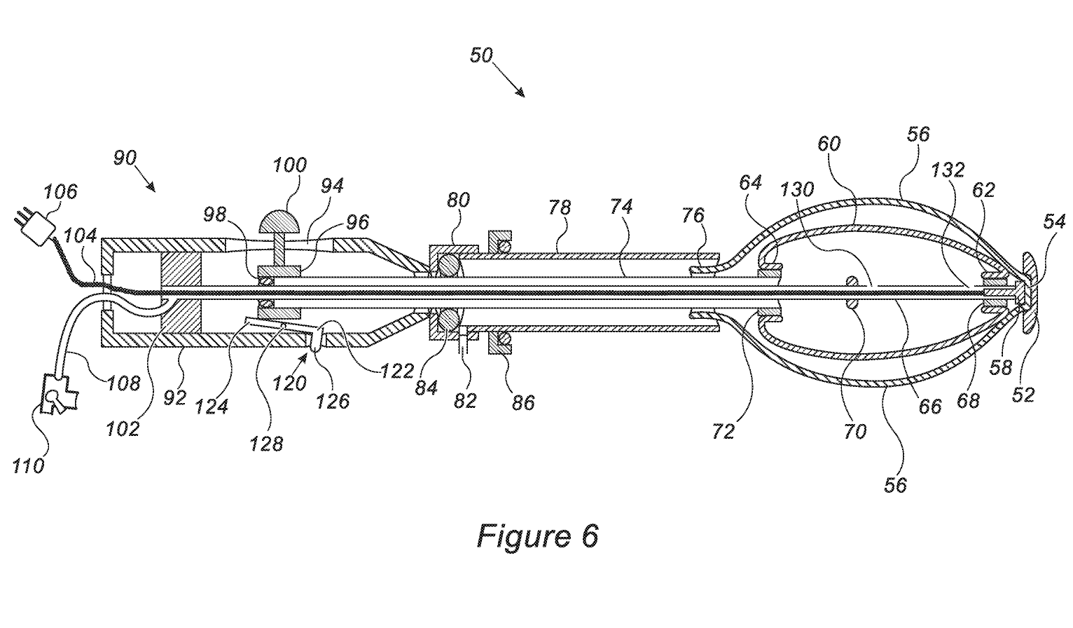

The far field bipolar technology described above, may be implemented using various methods and devices. Some such embodiments are described in FIGS. 6-8.

Flexcircuit Design.

FIG. 6 is a simplified longitudinal cross section of an embodiment of device 50, which may utilize a flexible PCB material to form the longitudinal electrode structure, and facilitate wiring of all electrodes.

From distal to proximal, FIG. 6 shows device 50 comprising atraumatic cap 52, flexcircuit plate 54, flexcircuit arms 56, tip plug 58, balloon 60 having distal balloon neck 62 and proximal balloon neck 64, inner shaft 66, distal ring 68, stopper 70, proximal ring 72, outer shaft 74, flexcircuit arms proximal ring 76, outer sheath 78, sliding valve 80 comprising sheath port 82 and valve seal 84, sliding stopper 86, handle 90 comprising housing 92, slot 94, outer shaft base 96, outer shaft seal 98, retraction knob 100, inner shaft base 102, wires 104, electric plug 106, inflation tube 108, stopcock 110, locking mechanism 120 comprising lever 122, tooth 124, release button 126, and hinge 128.

More particularly, inner shaft 66 may be fixed to handle 90 via base 102, and to flexcircuit plate 54 via tip plug 58. Wires 104 may run through the length of inner shaft 66, entering it through base 102, and exiting through tip plug 58, where wires 104 connect to flexcircuit plate 54. Wires 104 may be connected to electric plug 106 at their proximal end.

Inflation tube 108 may connect to the lumen of inner shaft 66 via base 102. Base 102 may be sealed around the entry points of wires 104 and inflation tube 108 using glue or sealant, as known in the art. The distal end of inner tube 66 may be sealed by tip plug 58, and glue or sealant as known in the art, while allowing passage of wires 104 to connect with flexcircuit plate 54. In this embodiment, Wires 104 may be fixed at their passage points into and out of inner shaft 66 lumen, making sealing around these points easy to achieve.

Inner shaft 66 may further comprise proximal openings 130 and distal openings 132 allowing inflation of balloon 60. Stopper 70 may be located proximal to openings 130 and 132, and may limit movement of outer shaft 74 over inner shaft 66.

Outer shaft 74 may be fixed to outer shaft base 96. Retraction knob 100 may protrude out of housing 92 via slot 94. Outer shaft 74 may be slideaby positioned around inner shaft 66, and may slideably pass out of housing 92 via the distal end of handle 90. Outer shaft seal 98 may allow sliding of outer shaft base 96 around inner shaft 66, while preventing leakage between them.

In some embodiments, a certain degree of leakage through outer shaft seal 98 may be permitted, so as to enable smooth movement of outer shaft 74 over inner shaft 66, as long as this leakage when balloon 60 is fully inflated, remains insignificant, e.g., up to ˜2 cc per minute.

Proximal ring 72 may be fixed to the distal end of outer shaft 74. Proximal balloon neck 64 may be fixedly connected to proximal ring 72, and distal balloon neck 62 may be fixedly connected to distal ring 68 of inner shaft 66. Both balloon necks may be sealed around these attachments.

Thus, pulling on retraction knob 100 may cause outer shaft 74 to move proximally in relation to inner shaft 66, resulting with longitudinal stretching of balloon 60, which may straighten it out, and reduce its outer diameter, enabling insertion at a small outer diameter. When base 96 passes tooth 124 of locking mechanism 120, tooth 124 may prevent distal movement of base 96, thus locking outer shaft 74 in this position.

Pressing release button 126 may cause lever 122 to rotate around hinge 128, releasing tooth 124 of locking mechanism 120 and allowing distal movement of outer shaft 74. This may typically be done prior to balloon inflation.

Following release of locking mechanism 120, outer shaft 74 may again be free to slide over inner shaft 66. Balloon 60 may then be inflated via the lumens of stopcock 110, inflation tube 108, inner shaft 66, and openings 130 and 132. Inflation of balloon 60 may cause it to expand, shorten, and pull outer shaft 74 distally as it inflates.

The distal ends of flexcircuit arms 56 may be fixedly attached to the distal end of inner shaft 66 as they may be continuous with flexcircuit plate 54, which may in turn be connected to tip plug 58, which may be connected and sealed to the distal end of inner shaft 66.

In contrast, the proximal ends of flexcircuit arms 56 may be connected together at flexcircuit arms proximal ring 76, which may be slideably positioned around outer shaft 74. Although in the current embodiment, device 50 may typically be configured to be used with a balloon inflation volume of ˜170 cc, the length of longitudinal flexcircuit arms 56 from flexcircuit plate 54 to proximal ring 76 may optionally be made sufficiently long to allow them to radially expand around a balloon inflated to any reasonable volume. For example, if balloon 60 were inflated to ˜400 cc (which may be considered a very high volume for this application), the length of longitudinal flexcircuit arms 56 from flexcircuit plate 54 to proximal ring 76 may be at least ˜14.4 cm, whereas for a 170 cc balloon volume, a length of ˜10.8 cm would suffice.

The structure described above may allow flexcircuit arms 56 to freely slide over outer shaft 74. Pulling retraction knob 100 proximally to stretch balloon 60, may cause proximal ring 72 of outer shaft 74 to pull flexcircuit arms proximal ring 76 in the same direction, thus also stretching and flattening out the longitudinal electrode structure formed by flexcircuit arms 56.

During inflation of balloon 60, flexcircuit arms proximal ring 76 may be free to slide along outer shaft 74 as it is pulled distally by the expansion of balloon 60, allowing flexcircuit arms 56 to expand radially, separately from balloon 60. This is especially important in case balloon 60 is made of a compliant material, whereas flexcircuit arms are made of a non-compliant material, which might create a different degree of stretch for each of them at various points along their circumference.

In case balloon 60 is made of a non-compliant or semi compliant material, separate expansion of the balloon and flexcircuit arms 56 may still be advantageous, as folding of balloon 60 may require it to assume a configuration that cannot be achieved if connected to flexcircuit arms 56. Alternatively or in combination, focal connections between balloon 60 and flexcircuit arms 56 at specific locations, may be desirable.

As described above, inner shaft 66 may be fixedly connected to handle 90, and outer shaft 74 may be slideable over inner shaft 66, but may be fixedly connected to retraction knob 100 which may slide within slot 94 of handle 90, thus preventing rotation of outer shaft 74. Therefore, both inner shaft 66, and outer shaft 74, maintain a constant orientation in relation to each other and handle 90. This can be important as it prevents unintentional twisting of the electrode structure which could interfere with retraction of the device. In addition, this arrangement allows the user to control the orientation of the electrodes, which is of significance in designs where there is asymmetry of the ablation pattern, as will be further described below.

In some embodiments, rotation of the flexcircuit arms proximal ring 76 may further be prevented, for example by making outer shaft 74 and flexcircuit arms proximal ring 76 include a directional feature such as a non-circular cross section, as known in the art. This is a further measure preventing twisting of electrodes.

Alternatively, a similar result may be achieved by providing a longitudinal slot along inner tube 66, and a protrusion from outer tube 74 which may slide inside said slot. Such an arrangement may require creating a seal between outer shaft 74 and inner shaft 66 distal to said slot to prevent leakage, for example by moving outer shaft seal 98 to the distal end of outer shaft 74.

Sheath 78 may be slideably positioned over outer shaft 74, with valve 80 creating a seal between them. Valve 80 may be any suitable valve which can allow both a good seal and sliding between the elements, as known in the art, for example a Tuohy-Borst valve. Sheath 78 can be moved distally to cover flexcircuit longitudinal arms 56, balloon 60 and inner shaft 66, in the folded or compressed position of device 50. In the fully folded or compressed state, the distal end of sheath 78 may be flush with the proximal end of atraumatic cap 52.

Atraumatic cap 52 may typically comprise a rounded structure, with smooth edges. Although it may be dome shaped, it may typically be rather flat, having a thickness of approximately 3-5 mm or less so as to prevent pushing of the tissue away from the electrodes. Its edges may optionally extend laterally to cover a part of or the entire distal tip of outer sheath 78. It may be made of plastic, rubber, metal, or any other biocompatible material, and may be glued, welded, screwed, or otherwise attached to the distal end of ablation device 50. In some embodiments, atraumatic cap 52 may merely comprise a layer of adhesive or other type of coating applied to the distal end of ablation device 50, typically to flexcircuit plate 54. In yet other embodiments, atraumatic cap 52 may be comprised of flexcircuit plate 54 alone. In yet other embodiments, atraumatic tip 52 may be made of gel, which may optionally dissolve following insertion into the body or following contact with fluid.

Sliding stopper 86 may comprise an element which can be easily slid along sheath 78 and locked at any position as desired by the user, to limit the depth of insertion into the body to the pre-measured urethral depth or desired deployment depth.

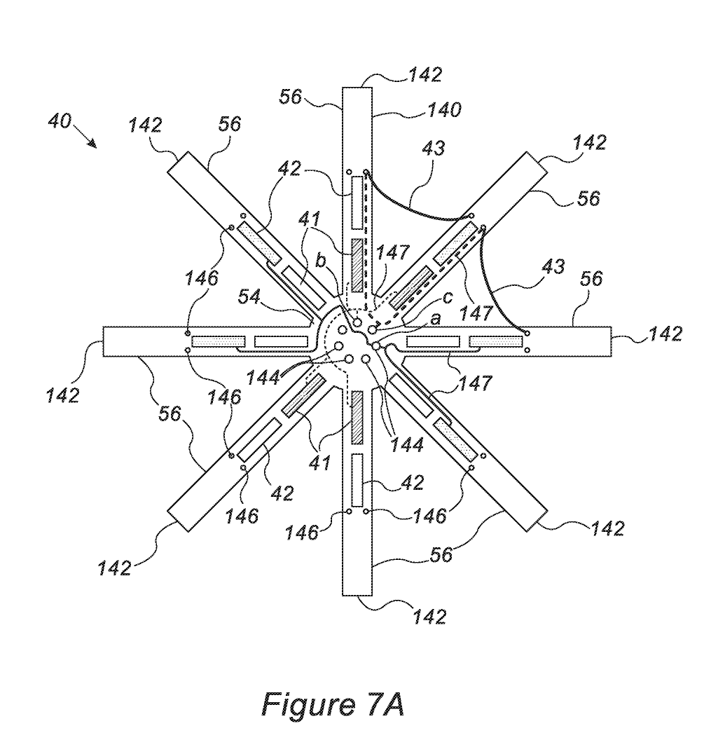

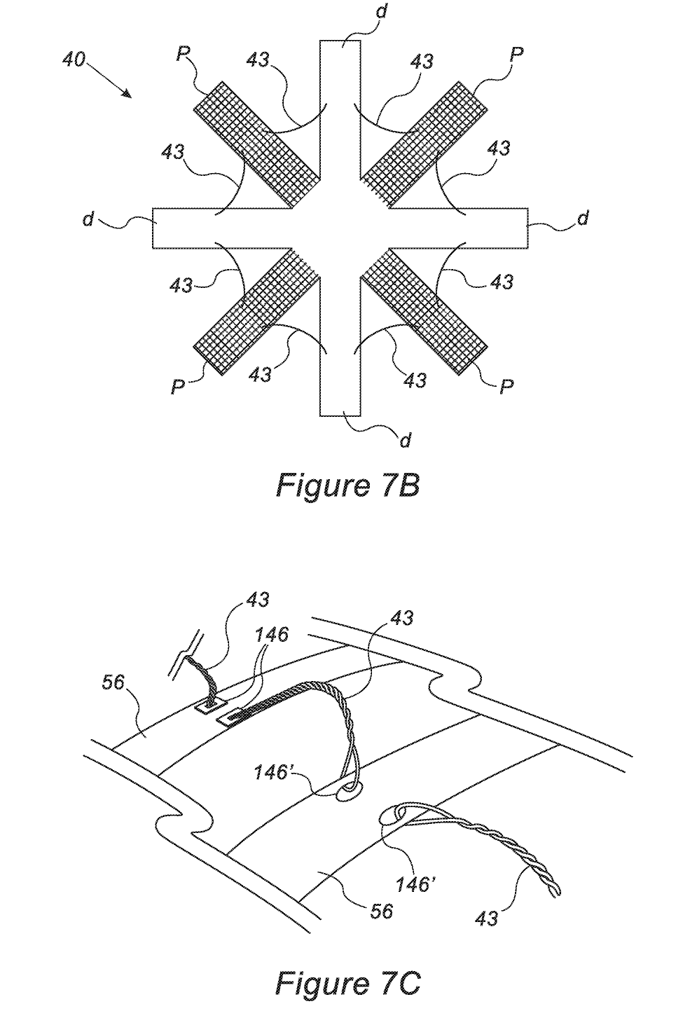

FIG. 7a describes the electrode structure 40 of an embodiment of device 50.

More particularly, FIG. 7a is a schematic top view of electrode structure 40, similar in general to that shown in FIG. 3. Electrode structure 40 may comprise a flexible PCB 140, including flexcircuit plate 54, flexcircuit arms 56, having proximal ends 142, distal connectors 144, proximal connectors 146, distal longitudinal electrode segments 41, proximal longitudinal electrode segments 42, and insulated tracks 147. Electrode structure 40 may further comprise circumferential electrode segments 43, typically made of wires, braids, or other, preferably flexible, conductive material.

In the interest of clarity, only some of the insulated tracks and circumferential electrodes are shown. It should be understood however, that typically all flexcircuit arms may have electrode segments 41 and 42 on them, and circumferential electrode segments 43 between them, with connections as needed made by insulated tracks 147, which may run in different layers of the PCB.

It should also be noted that the length of circumferential electrode segments 43 may typically be shorter than appears in FIG. 7a, due to its being a two dimensional representation of a three dimensional structure.

Returning to the PCB, flexcircuit plate 54 may serve as the base for electrode structure 40 of the current embodiment, and wires 104, which may run through inner shaft 66 and may be connected to it, typically by soldering wires 104 to distal connectors 144.

Flexcircuit longitudinal arms 56 extend radially from flexcircuit plate 54. Typically, there may be eight arms 56 but this number can vary from 1 to approximately 20.

As shown in FIG. 7a, several electrode segments of each type may be connected via insulated tracks 147 to each other and to at least one distal connector 144. Typically, each set of four distal longitudinal electrode segments 41 working as one pole may be connected to one distal connector 144, each set of four proximal longitudinal electrode segments 42 working as one pole may be connected to another distal connector 144, and each set of two circumferential electrode segments 43 working as one pole may be connected to yet another distal connector 144.

For example, to drive the longitudinal electrodes as shown in FIGS. 5A-5B, two distal longitudinal electrode segments 41 on adjacent flexcircuit arms 56, and two on the opposing arms may be connected to the same distal connector 144, labeled “a”, which may be fed by one wire 104, forming one pole, while two proximal longitudinal electrode segments 42 on adjacent flexcircuit arms 56, and two on the opposing arms, may be connected to another distal connector 144, labeled “b”, which may be fed by another wire 104, forming the other pole.

Continuing the same example, to drive the circumferential electrode segments as shown in FIGS. 5C-5D, two circumferential electrode segments 43 on adjacent flexcircuit arms 56, may be connected to the same distal connector 144, labeled “c”, which may be fed by one wire 104, forming one pole, while the two opposing circumferential segments 43 on adjacent flexcircuit arms 56, may be connected to another distal connector 144 (not shown), which may be fed by another wire 104, forming the other pole.

When fabricating the above described embodiment of device 50, wires 104 passing out of inner shaft 66 distal end, may be soldered to distal connectors 144 of flexcircuit plate 54, which may thereafter be connected to the distal tip of inner shaft 66, optionally using tip plug 58. Flexcircuit arms 56 may be bent parallel to the longitudinal axis of inner shaft 66, and flexcircuit proximal ends 142 may all be connected to each other at flexcircuit arms proximal ring 76, around outer shaft 74.

Circumferential electrode segments 43 may be soldered to proximal connectors 146, creating “bridges” between adjacent flexcircuit arms 56.

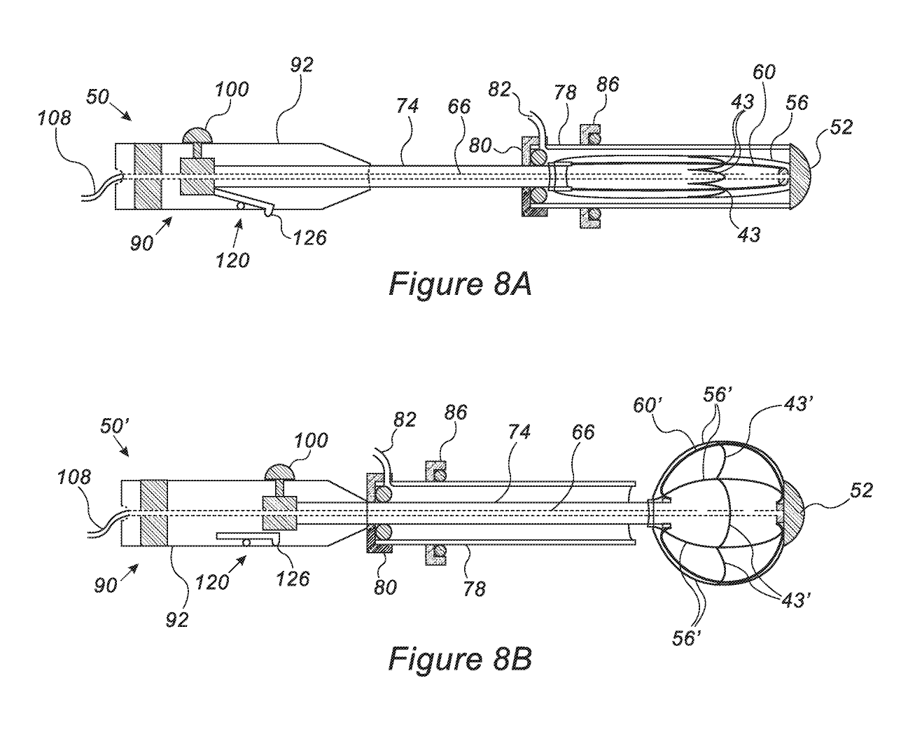

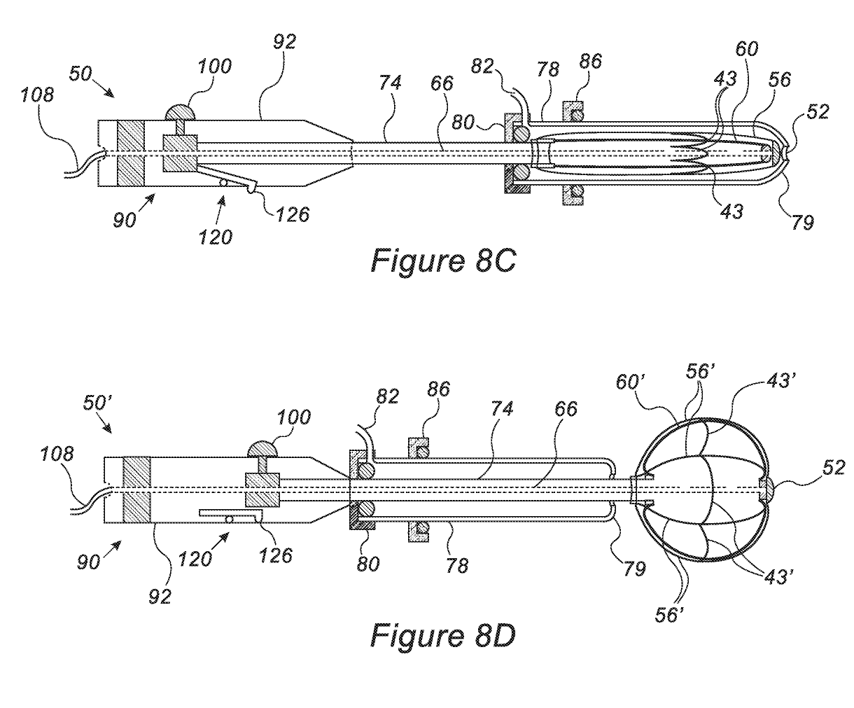

FIGS. 8A and 8B describe device 50 in its folded or compressed and fully deployed and inflated states, respectively.

More particularly, FIG. 8A is a simplified schematic longitudinal section of device 50 in its folded or compressed state 50.

From distal to proximal are seen: atraumatic tip 52, flexcircuit legs 56 and circumferential electrode segments 43 in their collapsed state, overlying deflated balloon 60, outer sheath 78 with sliding valve 80 and sheath port 82, and sliding stopper 86, outer shaft 74, inner shaft 66, handle 90 comprising housing 92, locking mechanism 120, release button 126, retraction knob 100, and inflation tube 108.

Circumferential electrode segments 43 are seen bent at their middle, both halves of each segment becoming parallel to the longitudinal axis of inner shaft 66, allowing the structure to fit inside sheath 78.

Locking mechanism 120 is shown holding outer shaft 74 in a proximal position, longitudinally stretching balloon 60. Balloon 60 with the electrode structure are shown collapsed and covered by outer sheath 78.

FIG. 8B is a simplified schematic longitudinal section of device 50 in its deployed inflated state 50′.

From distal to proximal are seen: atraumatic tip 52, flexcircuit legs 56 and circumferential electrode segments 43 in their expanded state, overlying inflated balloon 60, outer sheath 78 with sliding valve 80 and sheath port 82, and sliding stopper 86, outer shaft 74, inner shaft 66, handle 90 comprising housing 92, locking mechanism 120, release button 126, retraction knob 100, and inflation tube 108.

Locking mechanism 120 is shown in its released state, outer shaft 74 is now shown in a distal position, having been pulled distally by balloon 60. Outer sheath 74 is shown drawn proximally, with balloon 60 fully inflated and the electrodes structure expanded. Circumferential electrode segments 43 are seen straightened out almost completely, enabling creation of an ablation line around the equator of the bladder.

The longitudinal electrode segments 41 and 42 in the above embodiment may typically be made of exposed tracks of PCB material, typically measuring approximately 0.5 mm*25 mm, and made of copper or other electrically conductive material. Dimensions may typically vary between 0.2 mm*10 mm to 1 mm*50 mm.

The circumferential electrode segments 43 in the above described embodiment may be made of bare wire, copper or another electrically conductive material of 30 AWG (American Wire Gauge), i.e., approximately 0.25 mm in diameter, which may be soldered to the proximal connectors 146. Wires or braided cables made of different materials may be used for these segments, such as stainless steel, silver etc. or for example copper with gold or other plating. Wires of different gauges can also be used, typically this will be in the range of 28-32 gauge.

Use of device 50 to perform TBP is described below, referring to FIGS. 8A-8B.

After appropriate cleansing and draping, the urethral length, or desired deployment depth may first be measured using a Foley catheter, and local anesthetic may be instilled into the bladder. Sliding stopper 86 may be locked over outer sheath 78 at the corresponding distance from atraumatic tip 52. Valve 80 may be locked to prevent unintentional deployment of the device.

Folded or compressed device 50, as seen in FIG. 8A, may be inserted into the urethra until sliding stopper 86 reaches the external urethral meatus. Valve 80 may then be released.

While keeping sheath 78 in place so it does not move relative to the patient's body, handle 90 may be pushed forward, thus deploying the device, i.e. passing it out of outer sheath 78.

Release button 126 may be pressed to release locking mechanism 120. Balloon 60 may be inflated with fluid via inflation tube 108, causing outer shaft 74 to slide distally out of handle 90, and longitudinal flexcircuit arms 56 and circumferential electrode segments 43 to expand radially around balloon 60, as shown in FIG. 8B.

The bladder may be drained around the electrodes and balloon 60 through sheath port 82.

Measurement of impedances between the electrode sets may be performed, followed by energizing of the electrodes with an RF generator utilizing the far field bipolar technology, as described above, for ablation of the desired isolation lines pattern on the bladder wall.

Fluid may be instilled in the bladder around the balloon and electrodes via sheath port 82, optionally by transferring fluid from the balloon to the bladder, such that the bladder volume may be kept substantially stable during balloon deflation. This method may prevent interference with collapse of the balloon and electrode structure and removal of the device.

Retraction knob 100 may be pulled proximally to retract outer shaft 74, stretch balloon 60, and collapse longitudinal flexcircuit arms 56 and circumferential electrode segments 43. Once pulled sufficiently proximally, locking mechanism 120 may lock, and handle 90 may be pulled while holding sheath 78 in place, to retract balloon 60 with the electrode structure into sheath 78.

Handle 90 may then be pulled further proximally to remove device 50 from the patient's body.

Various modifications of the above described embodiments may be advantageous.

In some embodiments, the distal tip of outer sheath 78 may be modified to make it atraumatic.

For example, the distal tip of outer sheath 78 may be filed or otherwise processed using heat or other methods as known in the art so as to be extremely rounded and smooth, to prevent any damage to tissue during its insertion to a patient's body.

The distal tip of outer sheath 78 may additionally or alternatively be made soft, using similar processes. The transition from a more rigid to a softer consistency may occur gradually over a certain distance, typically along 2-20 mm.

Another possible embodiment of outer sheath distal tip 79 is shown in FIGS. 8c-8d, which are otherwise identical to FIGS. 8a-8b.

Outer sheath distal tip 79 may additionally be made narrower than the more proximal part of outer sheath 78, so that in the folded or compressed state shown in FIG. 8c, it may extend distally to and cover atraumatic cap 52 or flexcircuit plate 54. Outer sheath distal tip 79, although narrow, may be sufficiently flexible to allow passage of atraumatic cap 52, flexcircuit plate 54, electrode structure 40, and expandable element 30 distally during deployment as shown in FIG. 8d, and proximally during retraction.

In FIG. 7a, each circumferential electrode segment 43 is connected to and receives power from a proximal connector 146 on one “powered” flexcircuit arm 56 (i.e., may deliver power), and connects to another proximal connector 146 on an adjacent flexcircuit arm 56 which is “dead” (i.e., may not deliver power).

In some embodiments, power to circumferential electrode segments 43 may be delivered through only four of the flexcircuit arms 56, so that there may be a “powered” flexcircuit arm 56 between each two “dead” flexcircuit arms 56.

FIG. 7b is a schematic depiction of electrode structure 40 of such an embodiment. Flexcircuit arms 56 that are “powered” are marked “p” (and cross hatched), while flexcircuit arms 56 that are “dead” are marked “d”.

As can be seen, each pair of circumferential electrode segments 43 may receive power from one “powered” flexcircuit arms “p” between them, and each circumferential electrode segment 43 may connect to a separate adjacent “dead” flexcircuit arm “d”. Such an arrangement may simplify the PCB.

Use of copper wires for the circumferential electrode segments may provide the advantage of high conductivity, low cost, and ease of use.

However, these wires are malleable and tend to maintain the device in its open position even when the balloon is deflated, requiring pulling on the outer shaft, and retraction into sheath 78 to cause collapse of the electrode structure.

In addition, these wires are prone to breakage due to fatigue as a result of repeated bending at the same points. For disposable devices this is typically sufficient, however for a more durable design, any of the following modifications may be utilized.

A possible modification may include use of a braided cable or wire. A braid may be less easily fatigued, and depending on its material, may be non-malleable. For example, a stainless steel cable can be used, and if its conductivity is deemed too low, a silver wire or other highly conductive material can be incorporated in it to increase conductivity.

Connecting such a braid to proximal connectors 146 may require laser welding, as braided cables tend to act as wicks when soldered, resulting in hardened, fragile segments.

Typically, such soldering or welding to a “powered” proximal connector 146 may result with a durable attachment, as the electrical lead embedded within the PCB provides a good anchor. However, when connecting to a “dead” proximal connector 146, the attachment may be prone to detachment if the connector is a superficial solder pad on an outer layer of the PCB. This may be solved for example by providing proximal connectors 146 which may have a long enough extension embedded within the PCB, even though this lead may not connect to a power source (it is “dead”).

Alternatively, as shown in FIG. 7C, “dead” proximal connectors 146′ may comprise a hole in the flexiciruit arm. Circumferential electrode segments 43 may be passed through this hole, looped and twisted around themselves, then soldered to the “powered” proximal connectors 146 on the adjacent flexcircuit arm 56, as depicted in FIG. 7c.

Several possible modifications may utilize the same flexcircuit as described above for longitudinal electrode segments 41 and 42, instead of wires, for circumferential electrode segments 43.

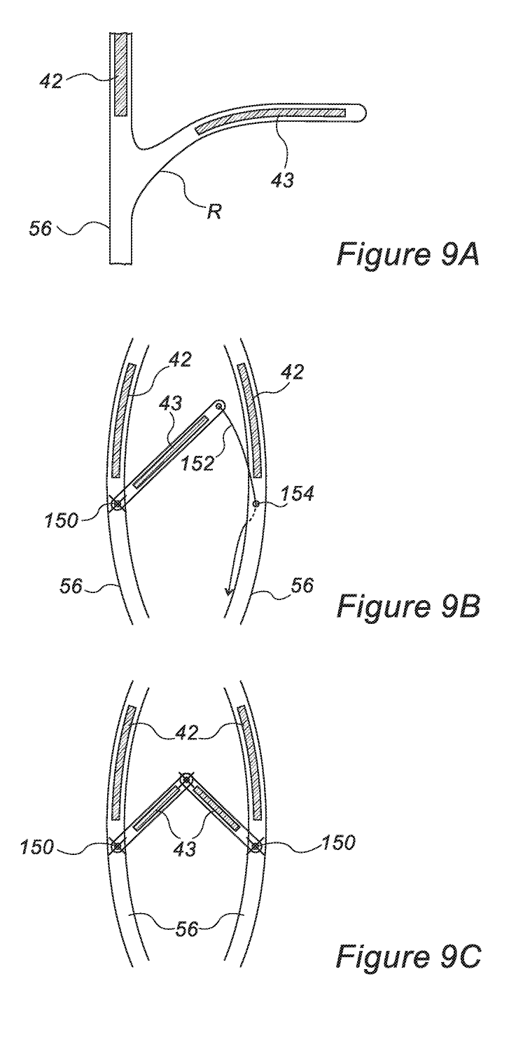

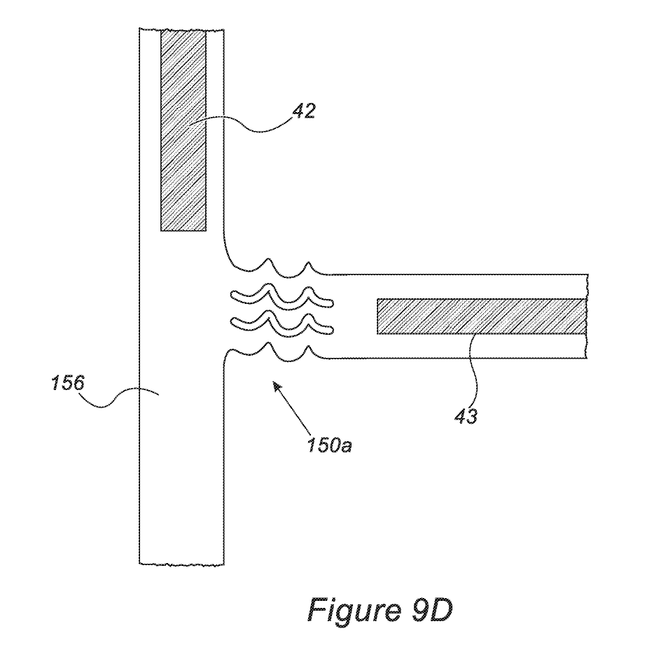

FIG. 9A shows an embodiment in which each circumferential electrode segment 43 may be a simple bifurcation off each flexcircuit longitudinal arm 56. In this embodiment, each circumferential segment 43 may be connected at only one side. When pulled into sheath 78, segment 43 folds and assumes a position parallel to arm 56 by moving out of the plane of PCB 140. Since this fold takes up significant volume, the bifurcation off each flexcircuit longitudinal arm 56 is positioned at a slightly different height along arms 56, so that each fold occurs at a different height inside the sheath, creating minimal overlap between these folds, thus enabling crimping into the small diameter of the sheath.

The radius of the bifurcation marked R, may be configured to facilitate entry of the circumferential segment 34 into the sheath when pulled into it during crimping.

FIG. 9B shows a different embodiment of circumferential electrode segment 43, in which segment 43 may comprise a separate strip of flexcircuit, rotatably connected to one flexcircuit arm 56 by flexcircuit hinge 150, and to an adjacent flexcircuit arm 56 by tightening wire 152 which passes through loop or hole 154 on said adjacent arm 56, and from there to handle 90. Following or during inflation of balloon 60, tightening wires 152 may be pulled proximally causing circumferential electrode segment 43 to extend between each two adjacent arms 56. Release of tightening wires 152, a sufficiently low friction between tightening wires 152 and loop 154, and a sufficient angle of segment 43 when at its deployed position may enable crimping back to the folded position after balloon deflation and pulling into sheath 78. An additional set of wires may optionally be connected to the tip of each segment 43, and passed through a second loop positioned at the distal end of each proximal longitudinal electrode segment 42, such that when pulled tight, this second set of wires may cause folding of the segments 43, causing them to become parallel to arms 56. In this embodiment, hinges 150 may serve both for enabling rotation of the segments, and for conduction of electrical current between them.

FIG. 9C is a similar embodiment, in which each circumferential electrode segment 43 may comprise two separate strips of flexcircuit, rotatably connected to each other by middle hinge 156, and to each of two adjacent arms 56 by flexcircuit hinges 150. This structure may straighten out as balloon 60 inflates, and may fold back (like scissors) following deflation and during pulling into sheath 78.

FIG. 9D shows an embodiment 150a of flexcircuit hinges 150 or middle hinges 156 in which the hinge may be created by cutting the flexcircuit in a “zig zag” pattern. This may enable bending at the cut area, and passage of conductors across the hinge through the PCB, which can consequently be printed as a single piece, eliminating the need for assembling many rotating hinges. These cuts may be used to facilitate the use of PCB bifurcations as the circumferential electrodes (as in FIG. 9C described above).

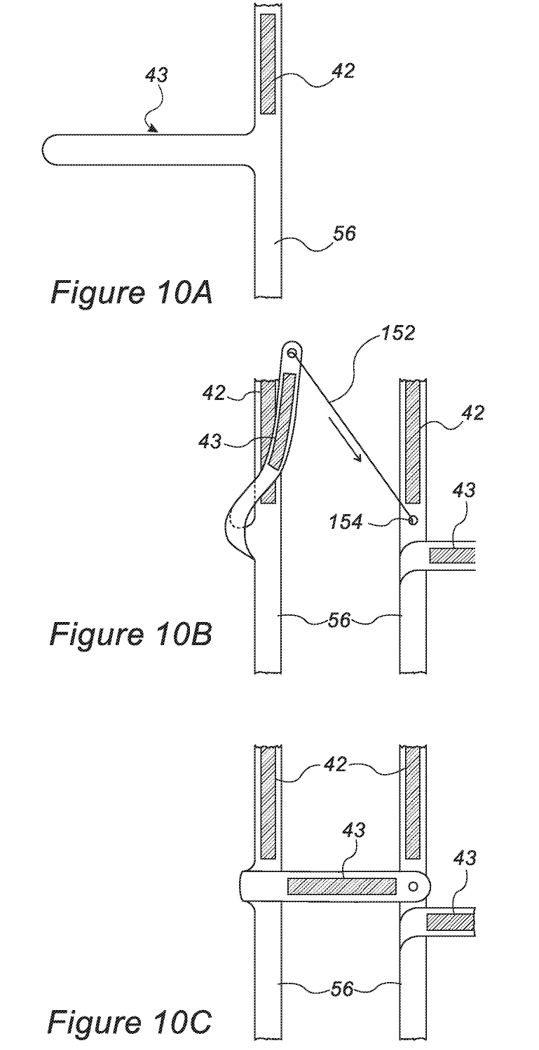

FIGS. 10A-10C show an embodiment similar to that shown in FIG. 9A, in which a tightening wire 152 from the end of segment 43, passed through loop 154, may be used as described for FIG. 9B above, for pulling segment 43 to its deployed position, following inflation of balloon 60. The current embodiment is different in that it is the back side of PCB 140 that is used as the actual exposed electrode segment 43. The advantage is that because it does not have a hinge, this segment 43 is most easily folded when folded or compressed by turning the back side of PCB 140 outwards, and in the currently described embodiment, deployment of this segment may be easier, as there is no need to change the side of the PCB facing outwards.

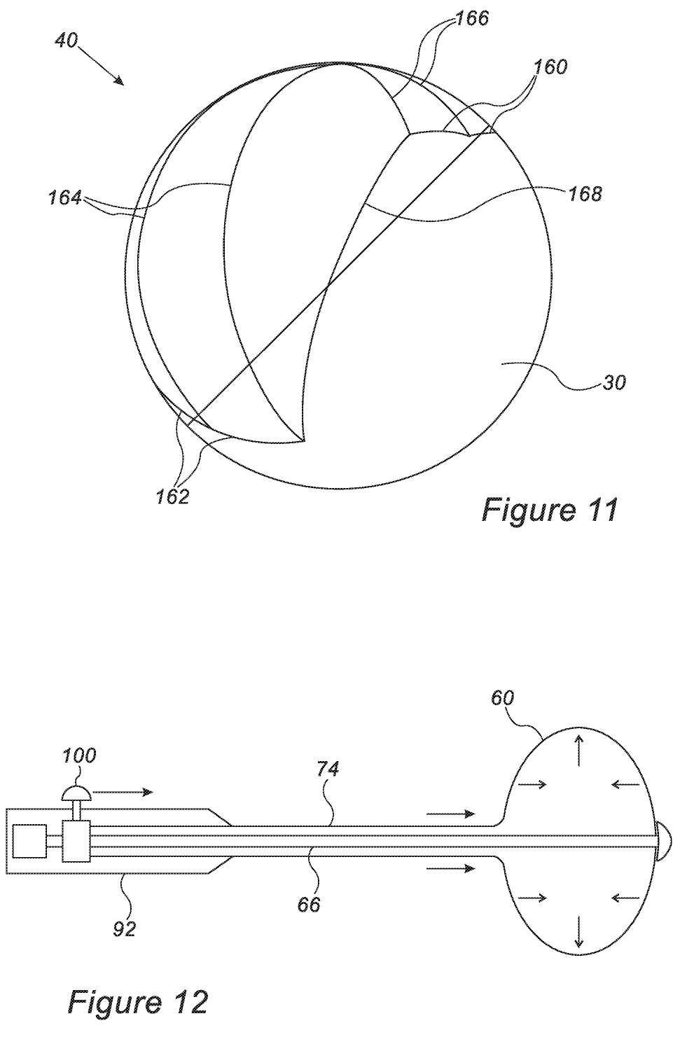

Note that the above described device 50 can be made with an asymmetrical electrode structure that, for example being slanted anteriorly, sparing the posterior aspect of the bladder from ablation as described above, while still using the far field bipolar technology, as long as equal lengths of opposing electrodes are used. FIG. 11 described such a possible design.

More particularly, FIG. 11 is a simplified schematic side view of an electrode structure 40 over a spherical expandable element 30, showing upper circumferential electrode segments 160, lower circumferential electrode segments 162, anterior longitudinal electrode segments 164, posterior longitudinal electrode segments 166, and diagonal electrode segments 168.

Upper circumferential electrode segments 160 and lower circumferential electrode segments 162 may be at the same distance from the equator line of spherical expandable element 30, and therefore may be of the same length, making them appropriate for use as bipolar pair. Diagonal electrode segments 168 are located one at each side of structure 40, and may also be a good bipolar pair.

Various combinations of parts of anterior longitudinal electrode segments 164 and posterior longitudinal electrode segments 166 may also be used as bipolar pairs, to enable creating this pattern using the far field bipolar technique.

In other embodiments, the far field bipolar may be used with opposing electrodes of unequal length. Rather, an equal degree of ablation at each electrode may be achieved by making the surface area of the electrodes equal (the shorter electrode being wider in an equal proportion).

In other embodiments, the far field technology may be used with a deliberate asymmetry between the two electrodes. This configuration may be useful when one electrode (or set of electrodes) is applied to a different surface, or at a different pressure. One example of this configuration may be coupling a relatively longer segment, apposed to the bladder dome, with a relatively shorter segment, apposed to the lateral wall of the bladder. This configuration may be useful for example when the contact pressure at the dome exceeds the contact pressure at the lateral walls (e.g., due to manual pressure applied along the long axis of the device, or other causes). Thus, the increased contact pressure at the dome may be offset by the decreased current density (due to the increased electrode length), and the resulting ablation may still be symmetrical though the electrode lengths and surface areas are different.

In other embodiments, the far field bipolar technology may be used with asymmetrical electrodes, with the intention to induce asymmetrical lesions. This configuration may be useful when different anatomical zones are ablated with coupled electrodes. An example of this configuration includes coupling longer distal (closer to the tip of the device) electrodes with shorter circumferential electrodes, with the intention to have increased current density and increased lesion depth at the circumferential electrodes. This configuration may be useful in creating shallower lesions in the anatomical regions of the bladder that are intraperitoneal, and deeper lesions in the regions that are not in direct contact with the peritoneal cavity. Another example may be coupling longer posterior lines with shorter anterior lines, in order to create differentially shallower lesions at the posterior side of the bladder, that is adjacent to sensitive organs such as the vagina (in women) and the seminal vesicles in men.

Yet another example may include a situation where the goal is creation of a significant lesion at one pole, and no lesion, or an insignificant lesion at the other, for example when localized treatment at a specific region is desired, as will be elaborated below.

Further Improvements and Variations.

Deployment Controls.

A useful modification of device 50 involves preventing the possibility of mistakenly inflating balloon 60 before locking mechanism 120 was released. Such inflation prior to releasing the locking mechanism could damage the balloon and/or electrodes.

Preventing inflation before release of the locking mechanism can easily be achieved for example by incorporating a safety valve near the proximal end of inner shaft 66, which closes when compressed by outer shaft base 96 when at its locked position, such that the lumen of inner shaft 66 remains closed as long as locking mechanism 120 is locked. Release of locking mechanism 120 may allow outer shaft base 96 to move distally, releasing this safety valve, and allowing inflation to be undertaken.

Another useful modification involves preventing the possibility of retracting the balloon and electrodes into sheath 78 before flexcircuit longitudinal arms 56 are pulled taut using retraction knob 100. Such retraction prior to the longitudinal flexcircuit arms being drawn taut might cause their compression by sheath 78, with a resulting outward protrusion which might interfere with device removal.

Preventing retraction before the longitudinal arms are drawn taut can easily be achieved for example by incorporating into handle 90 a lever that locks sheath 78 in place, until outer shaft base 96 reaches the locked position of locking mechanism 120.

Axial Force Application.

Application of axial force along the longitudinal axis of the device 50 during ablation may aid in improving the contact between the electrodes and bladder wall and achieving a more homogenous ablation pattern. This force may typically be 1-20 N, preferably 2-10 N.

Application of such axial force may be performed manually by the user.

Control over the force may be provided for example by incorporating a spring based gauge into the handle, optionally with an alarm that would set go off if excessive force were applied.

Another way for applying this axial force, is shown in FIG. 12. FIG. 12 is a simplified schematic longitudinal section of device 50. This device is identical to that described previously in FIG. 6, with the exception that stopper 70 may be removed or positioned more distally on inner shaft 66, and casing 92 and slot 94 may be fabricated longer, so as to enable a longer range of motion of knob 100 in slot 94.

With this device, as shown in FIG. 12, application of axial force to balloon 60 may be performed by pushing retraction knob 100 distally, which may cause shortening of balloon 60, and an increase of its axial diameter, thus also increasing the radial force applied in that direction, which may improve circumferential electrode contact with the bladder wall. The axial force may also improve tissue contact of the longitudinal electrodes located at the distal end of the balloon.

Contact Force Measurement.

Measurement of the actual contact force between the electrodes and bladder wall may be beneficial, and may, for example, be performed by placing a miniature sensor adjacent to, or on at least one or more of the electrodes.

Miniature sensors that could be used for this include for example force sensing resistors such as the 400 series made by Interlink Electronics of Camarillo, CA 93012, USA.

Pressure sensors such as the FISO-LS series Fiber Optic Micro-catheter Pressure Transducers made by Harvard Apparatus of Holliston, MA 01746, USA, may also be used for estimation of the contact force.

The current disclosure further describes another method to assess the electrode-bladder contact pressure. According to this method, the volume in the balloon may be continuously monitored, as well as the pressure in the balloon (preferably measured at the balloon itself). The volume/pressure graph achieved in clinical practice (“measured pressure”) is compared to the bench tests of the same balloon (“expected pressure”). For any given volume in the balloon, the difference between the measured balloon pressure and the expected balloon pressure—is the balloon-bladder contact pressure.

The balloon may be inflated, pushed or deformed, until the desired contact pressure is achieved.

Pre-Choice of Optimal Balloon Volume.

In some embodiments described previously, the longitudinal flexcircuit arms 56 may be of variable length, and the balloon can be inflated to various volumes. The length of the circumferential electrodes may be variable as well (as previously described), or fixed to be long enough to accommodate the entire range of balloon inflation (being somewhat folded when the balloon is not fully inflated). In some embodiments, prior to insertion of the device, the bladder volume and pressures may be measured (as in urodynamic studies), and the volume of the bladder that achieves the desired contact pressure can be noted. Optimal contact pressure may typically be in the range of 5 cm H₂O to 100 cm H₂O, preferably 10 cm H₂O to 40 cm H₂O. Once this volume is noted, the device may then be inflated to this volume, to achieve the desired contact pressure.

Additionally, the volume of the balloon may need to be measured and correlated with the volume defined by the deployed geometry of the longitudinal and circumferential electrodes so that the balloon will better fit within this deployed geometry without placing added stress against the electrode elements or leaving void spaces without the desired pressure/force pressing the electrodes against the tissue.

Pre-Choice of Optimal Deployment Position.

In some embodiments, the balloon may have a fixed inflation volume. In these embodiments, the position of the balloon within the bladder (the displacement forward from the bladder neck) may affect the electrode-bladder contact pressure. In some embodiments, the optimal displacement may be predefined according to imaging of the bladder when filled with a fluid. When the optimal bladder pressure is reached, the long axis of the bladder may be measured, and the device may then be deployed in a position that will force the bladder to assume the same measured length. For example: a bladder may be filled with fluid, until a pressure of 40 cm H₂O is reached. The bladder's long axis may then be measured (for the purpose of this example it will be assumed to be 12 cm). Then, the device may be introduced and deployed so that the tip of the device (once inflated) may be 12 cm from the bladder neck. In some embodiments, clear markings on the device shaft may allow the user to clearly see how far in the device is.

Another advantage of choosing the optimal deployment position may be to make sure the trigone area is avoided. If the bladder size allows (as assessed by imaging, urodynamic study or cystoscopy) a deployment position can be chosen to make sure the trigone and the intradetrusor segment of the ureters are spared of ablation.

Automatic Controls

Further modifications of device 50 may involve automatic control of many of its functions and controls. This automatic control may possibly and preferably be executed by the controller/generator unit.

The controlled phases and actions may include any or all of the following as well as additional features that are not listed herein:

Automatic deployment (moving shaft out of sheath)—can be implemented using a linear or other electric motor that may be operated by the controller following insertion of the probe into a patient's urethra, and pushing a button, or activating a footswitch, by the user. This motor could for example push handle 90 distally relative to sheath 78. The controller may measure the force applied by the motor to ensure excessive force is not exerted on patient's tissues.

Automatic Release of Retraction Knob.

This may be achieved mechanically as described above, or electronically once a sensor detects full deployment was reached.

Automatic Inflation of Balloon—may be performed by the controller by activating an electronic pump, or opening an electronic valve to allow flow of fluid or gas at a known pressure. Pressure, rate of flow and total volume delivered may be monitored and controlled. In some embodiments, rapid balloon filling may be applied, to rapidly stretch the bladder and thus increase the contact pressure (before the bladder has sufficient time to relax into the new increased volume).

Automatic Application of Axial Force.

Axial force may be applied to the inner shaft 66 as described above, with the difference that this force may be applied automatically by the controller. For this purpose, the position of handle 90 relative to the patient may need to be controlled by the controller.

Alternatively or in combination, axial force may be applied to the outer shaft 74 of device 50 as described above, with the difference that this force may be applied automatically by the controller. For this purpose, the position of handle 90 relative to the patient may need to be controlled by the controller, by the user, or it may be fixed in space, while the controller may operate a motor that may push retraction knob 100 distally on handle 90. The force may be monitored and controlled by the controller by adjusting operation of the motor to keep the force within the desired range.

Automatic adjustments according to measurement of contact force—measurement of the electrodes-bladder wall contact force may be performed as described above, and monitored by the controller. These data as well as additional data such as impedance measurement may be used separately or together, to adjust the axial force, inflation volume or pressure, or ablation power, time, or other parameters affecting ablation results.

Such adjustments may optionally be performed based on comparison of the above measurements with a database containing historical data and appropriate treatment settings associated with them.

Automatic Balloon Deflation by Transfer of Fluid from Balloon to Bladder.

Operation of an electronic pump by the controller, optionally the same pump used for balloon inflation, can be automatically initiated to perform this fluid transfer. Alternatively or in combination, the tube leading to the bladder and the tube leading to the balloon may be connected to the same port, and a clearly marked valve may display and enable choosing the currently open path (balloon or bladder).

Automatic Collapse of Balloon and Electrodes.

Pulling the retraction knob proximally may be performed by a controller activated electric motor.

Automatic Retraction of Shaft.

Pulling handle 90 proximally relative to sheath 78 may be performed by a controller activated electric motor, optionally and preferably, the same motor used for automatic deployment.

Automatic Cessation of Ablation if Peak Temperature is Exceeded.

In some embodiments, the device may not have temperature sensors to allow control over ablation heat, and limiting the ablation temperature may be achieved by the balloon itself. In some embodiments, the balloon material and wall width is selected so that the balloon tears when in contact with heat beyond a certain temperature threshold. Rupture of the balloon may immediately and automatically reduce the ablation at that point by reducing the contact pressure and by flushing with fluid from the balloon. Additionally or alternatively, a pressure sensor may sense the reduction in balloon pressure (or the loss of balloon volume) and automatically abort ablation. In some embodiments the balloon material is polyurethane, and the wall thickness at the target volume is 0.02-0.005 mm, intentionally making the balloon likely to rupture when heated above 70 degrees Centigrade, thus aborting the procedure.

Means for Improving Electrode-Tissue Contact

Ensuring good mechanical and electrical contact between the electrodes and tissue may be crucial for achieving satisfactory ablation results. Various optional aspects of the device intended to improve this contact are described:

Fluid Removal

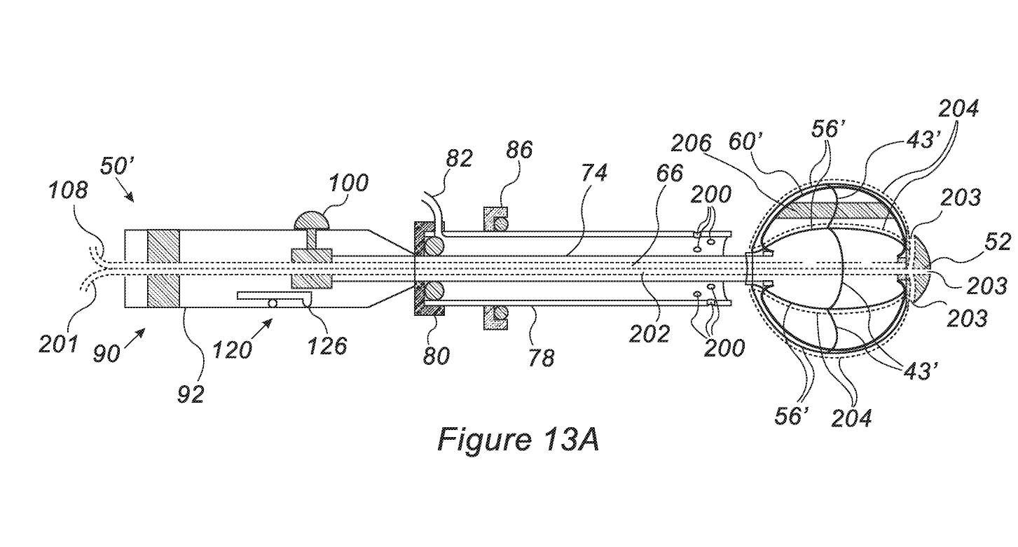

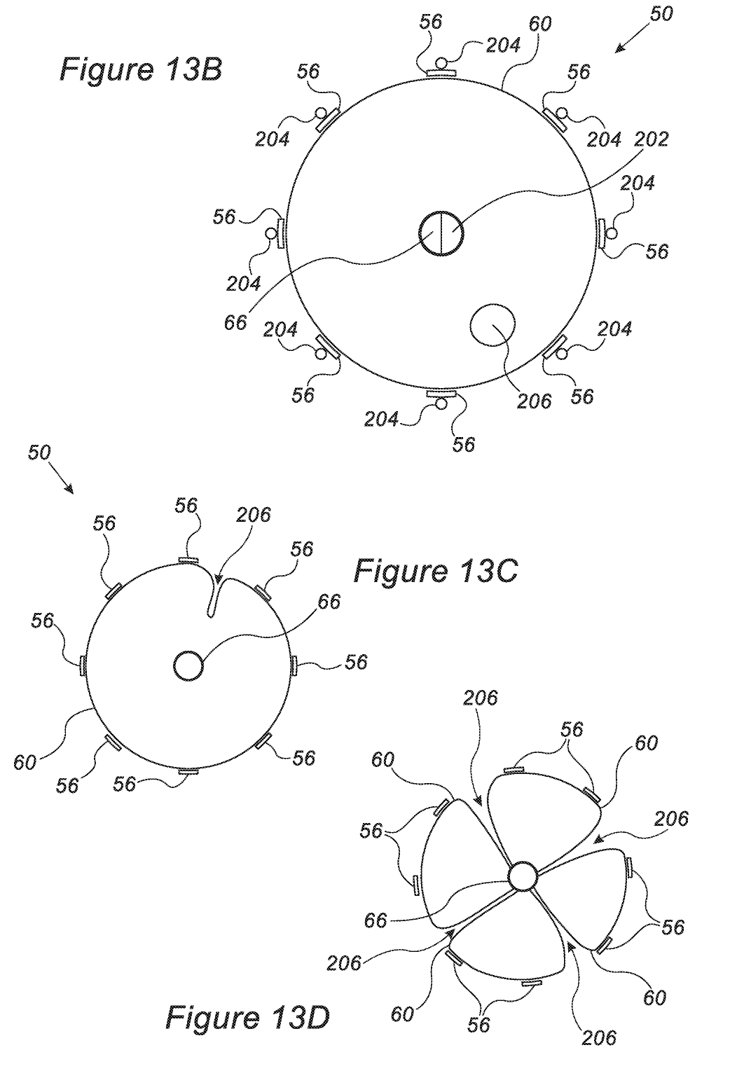

Following deployment and expansion of expandable element 30, excess fluid between the electrodes and the organ wall may be removed to improve electrode-tissue contact. This may be done by applying suction to the space between the electrodes and organ wall. Such suction may be applied using any one (or a combination) of the following means, shown in FIGS. 13a-d. FIG. 13a is a schematic longitudinal cross section of ablation device 50, while FIGS. 13b-d are axial cross section of device 50 at the level of the line marked Q in FIG. 13a. Apart from the information added in the following paragraphs, FIG. 13a is identical to FIG. 8b.

a. In some embodiments, application of suction to outer sheath 78, for example via sheath port 82, may easily remove fluid from at least the proximal area of the organ. Addition of sheath suction holes 200 around the distal end of outer sheath 78 may further improve the ability to apply suction via outer sheath 78, and reduce chances of tissue clogging its openings.

b. In some embodiments, suction may be applied to the distal end of device 50, for example, through a separate distal suction lumen 202 extending along inner shaft 66. Wires 104 may optionally pass through said separate distal suction lumen. Distal suction lumen port 201 may be provided at the proximal end of device 50, and at least one distal suction opening 203 may be provided at the distal end of device 50.

c. In some embodiments, suction may be applied around expandable element 30, for example through flexcircuit arms 56. This may for example be accomplished by flexible PCB 140 having fluid channels 204 over at least part of its external surface. For example, PCB 140 may comprise miniature tubes 204 along at least some of flexcircuit arms 56, which may for example extend from flexcircuit plate 54 all the way to flexcircuit arms proximal ends 142, or only along part of this length. Miniature tubes 204 may be connected to a suction source such as distal suction lumen 202, which may be connected to the tubes for example at flexcircuit plate 54. Miniature tubes 204 may have openings along their length to enable suction from various areas around balloon 60.

FIG. 13b is an axial cross-section of device 50 showing distal suction lumen 202 and inner shaft 66 in the center of inflated balloon 60, surrounded by eight flexcircuit arms 56, with a miniature tube 204 on each of them.

Alternatively, open channels, strips of an absorbant biocompatible cloth, or any other material, feature, or element capable of transmitting the suction may be used in place of miniature tubes 204.

d. In some embodiments, expandable member 30 may itself comprise at least one balloon channel 206 for transmitting suction. Such channel may for example be a tube like structure within the balloon, which may be part of the balloon and made of the same material as the balloon. The channels may connect an external source of suction with the balloon surface, or alternatively the channels may connect between different spaces around the balloon so as to allow suction or fluid to pass between them, such that suction applied at one area is transmitted to another area. For example, such a balloon channel 206, shown in FIGS. 13a and 13b, may connect the space between the treated organ wall and distal side of with the space proximal to the balloon. In another embodiment, balloon channel 206 may be at least one crease along the outer surface of the balloon as shown in FIG. 13c which is an axial cross-section of device 50. In yet another embodiment, balloon channel 206 comprises a space between two parts of one or more balloons 60 comprising expandable element 30 as shown in FIG. 13d, which is an axial cross-section of device 50.

e. Creating a vacuum (low pressure) within the bladder. In some embodiments, electrode-tissue contact may be improved by reducing the pressure inside the bladder by aspirating the liquids and gases from the bladder. To do so, means may be provided ensuring adequate seal around outer sheath 78 and the urethra or other tissue through which the device may be inserted, and seals between outer sheath 78 and internal device components (e.g. valve seal 84 may seal between outer sheath 78 and outer shaft 74) and between inner device components themselves (e.g. outer shaft seal 98 may seal between inner shaft 66 and outer shaft 74) such that fluid and gas leakage to the outside may be restricted.

Once adequate seal is ensured, aspiration of the fluids and gases inside the bladder may cause the pressure within the bladder to be less than the normal hemostatic pressure in the bladder and less than the pressure in the balloon when inflated, which may improve contact against the electrode elements.

Intentional Increase of Intra-Abdominal Pressure.

In some embodiments, electrode-tissue contact may be improved by increasing intra-abdominal pressure. This may be a transient increase prior to, or during the procedure, or both, or a longer lasting increase which may be applied for at least the duration of the procedure.

Such an increase in intra-abdominal pressure may be induced in many ways.

For example, in a conscious patient, it may be achieved by having the patient cough or perform a Valsalva maneuver.

In a ventilated patient under generalized anesthesia, this may be achieved for example by modifying ventilation parameters, such as increasing ventilation volume or positive end expiratory airway pressure.

A user, typically the treating physician, may increase the patient's intra-abdominal pressure by applying manual pressure on the patient's abdomen.

In some embodiments, a combination of the above may be used.

In some embodiments, intra-abdominal pressure may be monitored, for example using a rectal pressure probe, and a feedback indication may be provided to the physician and/or patient, to control the amount of pressure.

In some embodiments, the measured intra-abdominal pressure may be used to ensure that ablation may only be performed while the pressure is within a specific range, and may be automatically aborted if higher or lower than this range.

Micro-Conforming Electrodes.

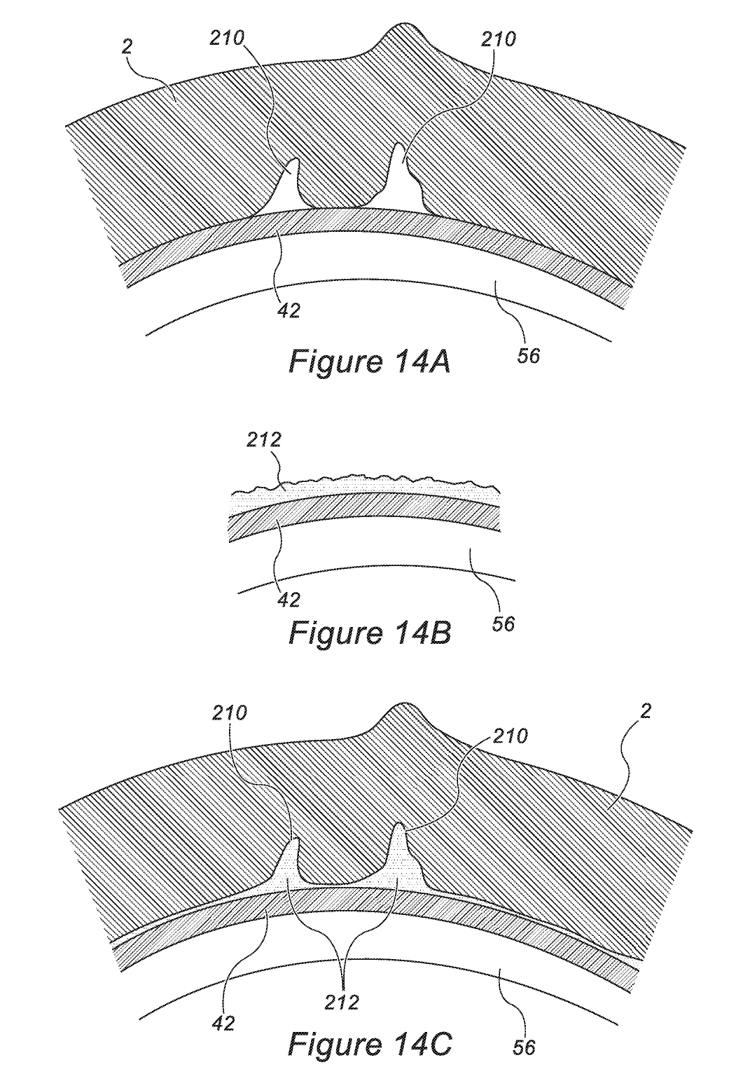

In some situations, despite overall good contact between the electrodes and organ wall, and even following the measures described above, there may still be small gaps remaining between the electrodes and tissue, in at least some points along the electrodes. As shown in FIG. 14A, this may for example be caused by small folds 210 in the organ wall 2, which may be due to incomplete stretching of the wall or other reasons. Furthermore, some organs may normally comprise structures that may cause small gaps, for example, rugae in the urinary bladder, or crypts and villae in the intestines. Changes in consistency, structure, or dimensions along the organ wall may be another reason for gaps. Gaps may be miniature, i.e. have dimensions on a magnitude of a few millimeters, or microscopic, i.e. on the magnitude of several microns. FIG. 14A is a schematic longitudinal section through flexcircuit arm and electrode segment 42 showing gaps 210 between organ wall 2 and electrode segment 42.

In order to ensure creation of continuous lesions along the electrodes despite such gaps, various modifications to the electrodes may be used that may improve contact in these situations.

For example, as shown in FIG. 14b, electrodes may be provided that have a conductive flexible or gelatinous material layer 212 on their surfaces. As shown in FIGS. 14c, material 212 may conform to the organ wall surface, such that it is compressed where the tissue protrudes, and squeezes into gaps 210, whether they are on the order of microns or millimeters in dimensions, thus improving the electrical contact between electrode segments 42 and tissue 2.

An example of a possible material 212 may be a poly (3,4-ethylenedioxythiophene)/polyurethane-hydrogel hybrid described by Sasaki et al. (Highly conductive stretchable and biocompatible electrode-hydrogel hybrids for advanced tissue engineering. Adv Healthc Mater. 2014 November; 3(11):1919-27).

Such a material may optionally be applied to the electrodes during PCB manufacturing, or manually during preparation of the probe just before the procedure.

Other materials or structures that may provide the same function as the above hydrogel by micro-conforming to the tissue surface may be used.

For example, a conductive fabric made of microfibers that constitute the electrodes or protrude from the electrode surface, may also provide this function. Such microfibers may be soft and conform to the tissue surface in a manner resembling the hydrogel described above. Alternatively, microfibers may possess sufficient sharpness and axial rigidity so as to penetrate the tissue, typically to a shallow depth limited to no more than 1 mm.

Semi-Conductive Medium.

Yet another embodiment intended to solve the problem of small gaps between the electrodes and the tissue relates to the medium used between the electrodes and organ wall. Typically such medium may be a fluid. Previous disclosures have discussed use of media that are either conductive such as saline or non-conductive such as glycine.

A highly conductive medium such as normal saline or hypertonic saline, may have the advantage of improving electrical coupling between the electrodes and tissue however it may run the risk of creating a “short” between the bipolar electrodes, if a thick enough layer of fluid is left between the electrodes and organ wall. In addition, it reduces the ability of the user to identify the existence of such excessive fluid layer, as it will not cause a noticeable change in impedance measurement between the electrodes.

A non-conductive medium such as glycine or sorbitol may have the advantage that if mechanical electrode-tissue contact is suboptimal, it may create a significant increase in measured impedance between the bipolar electrode pairs, so that the user may identify and fix the situation, for example by any of the above described methods.

The current invention includes optional use of a medium that may have a conductivity between that of the treated tissue and a complete insulator. For example, for a treatment in the urinary bladder, such a medium may be 0.1% saline, which may have a specific conductivity of approximately 0.1 Siemens/m. This conductivity is lower than that of the urothelium, which is described in various sources as having a conductivity between 0.2-1.9 S/m, yet it does not constitute a complete insulator. Thus, if a thin layer of this fluid remains between the electrodes and bladder wall, no “short circuiting” or “bypassing” of current flow through the tissue will occur, and an increase in impedance measurement may be detected, indicating the need for removal of fluid. On the other hand, fluid filling small scale or microscopic gaps within tissue folds or rugae along the electrodes may not act as an insulator, and may actually allow current flow, resulting with a more continuous lesion.

In some embodiments, a pressure sensor measuring balloon inflation pressure may be used during the procedure to detect various conditions, including leakage from the balloon. Typically, if measured proximal to inner shaft 66 or inflation tube 108, pressure may rise steeply during inflation due to resistance of the fluid pathways. Pressure may then decrease within a few seconds and stabilize at a pressure reflecting the fluid volume within the balloon, balloon size and elasticity, and bladder (or other treated organ) size and compliance.

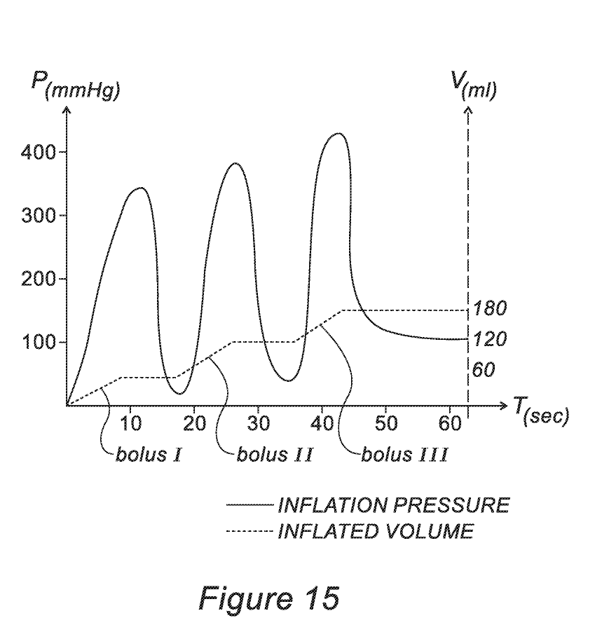

FIG. 15 is a schematic graph showing a possible inflation curve produced using such means. The horizontal axis represents time in seconds, the left vertical axis represents inflation pressure in mmHg, and the right vertical axis represents volume inflated into the balloon, in milliliters. In the depicted example, fluid is inflated in three boluses of ˜60 ml each. Measured pressure increases to ˜400 mmHg during active inflation, and falls to much less as it equilibrates with balloon pressure. Following the first fluid bolus, equilibrium pressure is close to zero, increases to 5-20 mmHg after the second bolus, and to approximately 90-150 mmHg after the third bolus. According to the authors' experience, most of this pressure reflects balloon pressure, as the bladder normally tends to relax and adds little to the overall measured equilibrated pressure.

Increases in pressure during bolus inflation to significantly more than ˜400 mmHg may typically indicate a problem such as inflation while the balloon is not fully deployed, a stuck electrode structure, a small or non-compliant bladder, or clogging of the fluid pathways. The user may choose to abort or change the procedure due to the above.

As mentioned previously, the pressure for the inflated device outside the body at a specific volume, may be pre-measured (“expected pressure”).

In case the equilibrated inflation pressure during the procedure is significantly higher (typically>˜20 mmHg higher) than the “expected pressure”, the user may conclude that the bladder is excessively small, contracted, or has very low compliance, and may choose to abort the procedure.

In case the measured inflation pressure does not equilibrate, or equilibrates to a level much lower than the pre-measured “expected pressure”, balloon leakage may be suspected, and the user may choose to replace the device.

It is important to note that this is only one of many possible inflation profiles. For example one that goes from flat pressure vs. volume, then monotonically increases in pressure with volume is possible as well.

Some embodiments may involve electrodes that may be printed over the expandable element.

The main advantage of such embodiments may be simplicity of manufacturing. Disadvantages may include the following:
First, if printed on a compliant balloon, such printed electrodes may change their electrical properties during inflation, thus making ablation results less predictable.
Second, such electrodes may be limited in the power they are capable of conducting.
Third, a balloon with printed electrodes may be more difficult to crimp into a small diameter.
Fourth, electrode printing technologies are not compatible with all elastomers, especially not those more resistant to heat, which may be more desirable in this application.

Non-Compliant Balloons and Methods of Manufacturing Thereof.

A non-compliant balloon appropriate for the device of the invention, may optionally be manufactured using blow molding, RF welding, or any other methods known in the art. Since the balloon of some of the embodiments may have an inflated diameter much larger than its proximal and distal neck diameters, standard blow-molding techniques may be less appropriate for manufacturing it as the initial tube will either need to be very thick, leaving very thick balloon necks, or it will not contain sufficient material for achieving a balloon wall of reasonable thickness in the inflated state.

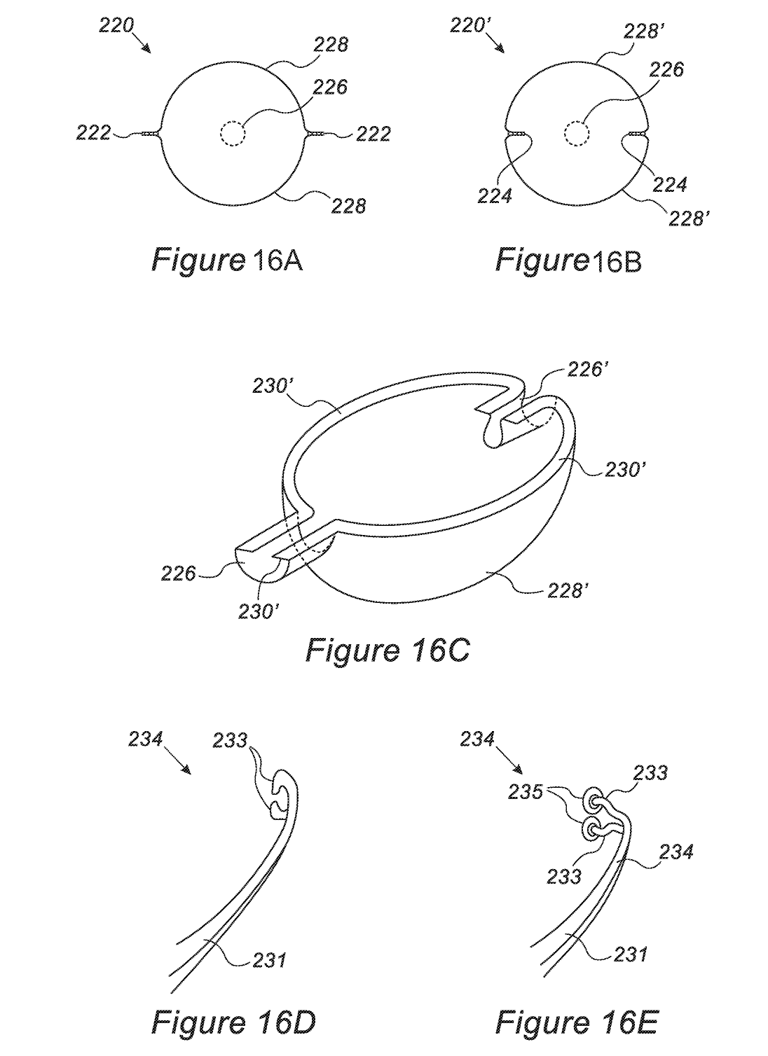

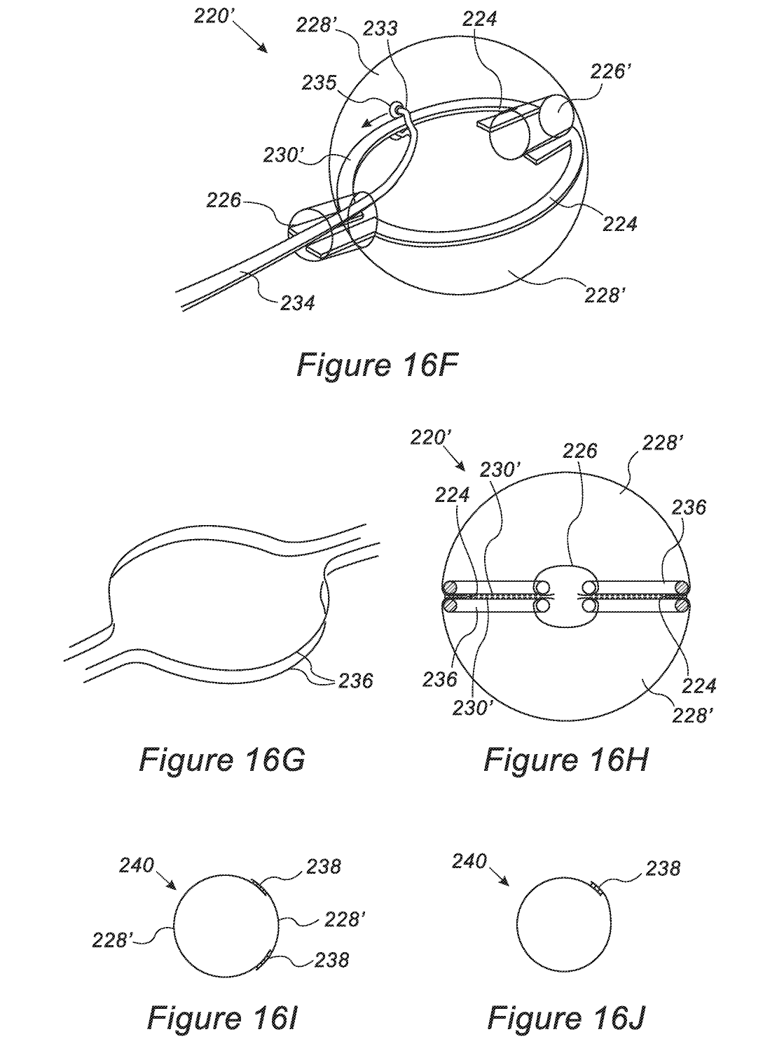

In such cases, RF welding (or laser, or ultrasound welding, or other form of connecting balloon parts) of a balloon made of at least two parts (typically two halves of the balloon) may be a suitable solution. A common result of such manufacturing method may be the production of a welded balloon 220 with an outwards facing seam 222 along the welding/connecting lines, as shown in FIG. 16a, which is a schematic axial cross-section of welded balloon 220 at a location similar to that marked by line Q in FIG. 13a, comprising two balloon parts 228. Also shown in FIG. 16a is balloon neck 226, although not on the section plane. Such an outward facing seam 222 may be undesirable for at least the following reasons: (1) the seam may enlarge folded balloon profile, (2) the seam may injure the inner surface of the treated organ, and (3) the seam may interfere with deployment of electrodes. Thus, manufacturing of a welded/connected balloon without an outwards protruding seam 222 may be desirable. Methods and apparatuses for manufacturing such a balloon 220′ are described below and in FIGS. 16b-i.

Welded Balloon with Inward Facing Seam.

In some embodiments, a welded balloon 220′ may be manufactured which may have an inward facing seam 224. FIG. 16b is a schematic axial cross-section of welded balloon 220′ a location similar to that marked by line Q in FIG. 13a, also showing inward facing seam 224, balloon neck 226, and balloon parts 228′.

Alternatively, the balloon may be manufactured with an outward facing seam in inverted upon itself resulting in the seam facing inwards.

FIG. 16c is a three dimensional sketch of balloon part 228′ which may comprise an inwards facing “flange” 230′ along its inner edges.

Balloon part 228′ may be manufactured by compression molding, injection molding, dipping, blow molding of a polymer sheet, or any other method known in the art.

Balloon parts may be brought together so that their “flanges” are adjacent each other. This may optionally be done inside a mold or jig to help approximate the parts.

Specialized “forceps” 232 or “rollers” 234 may be provided that may be passed through the balloon necks, and may clamp adjacent “flanges” together. In the following descriptions, forceps 232 and rollers 234 are used interchangeably.

Forceps 232 are shown in FIG. 16d, while rollers 234 are shown in FIG. 16e. Forceps 232 may typically comprise one or more elongate member 231, curved to one side, with a pair of apposing elements 233 at its distal end.

Rollers 234 have basically the same structure as forceps 232, with the addition of small wheels 235 on apposing elements 233.

These are just examples of possible tools, as any structure of a low profile that is capable of clamping together two balloon flanges and delivering the energy for welding them may be used. RF energy or other appropriate form of energy may be delivered between the two apposing elements 233 of “forceps” 232, or between an energy source positioned external to welded balloon 220 and “forceps” 232. Such energy may be RF energy, light (e.g. laser), ultrasound, heat, or any other energy as known in the art.

FIG. 16f is a three dimensional sketch of the manufacturing process of balloon 220′. Two balloon parts 228′ are shown held together with their flanges 230′ adjacent each other. Rollers 234 are seen traversing balloon neck 226, with apposing elements 233 and wheels 235 clamping flanges 230′ together, and welding them into inward facing seam 224.

In the embodiment depicted in FIG. 16f, rollers 234 may be used to weld a single point of the seam at each moment, and move along the seam to weld the whole length of the seam. The arrow denotes movement of rollers 234 towards balloon neck 226.

In some embodiments, a single pair of “forceps” 232 (or rollers 234) may be used. In some embodiments, at least two pairs of “forceps” may be used in parallel (e.g. for a balloon made of two halves, one pair may be used for each seam on either side of the balloon, preferably moving along the seams in parallel so that the balloon's symmetry is preserved. Additional pairs of “forceps” may be used, for example four pairs, two inserted and removed from either neck of the balloon.

Of note, balloon 220′ shown in FIG. 16f has one balloon neck 226 facing outwards, and one neck 226′ inverted inwards (typically at least the distal balloon neck would be inverted inwards). Regardless, the balloon of the device may have both necks facing outwards or inverted inwards, and may still be manufactured by any of the above methods. Alternatively, the balloon may be manufactured with the necks protruding outwards, and the necks may be inverted inwards in a post manufacture process.

In some embodiments, instead of facing inwards, “flange” 230′ may be at any angle to the balloon part wall, e.g. tangent, facing outwards, or any other angle. In such embodiments, the “forceps” 232 may also invert the “flange” 230 so as to cause it to face the opposite flange of the adjacent balloon part, just prior to welding.

In some embodiments, the balloon parts' “flanges” 230′ may be brought together from the inner side of the balloon by “clamps” 236 that may be designed to hold together the whole seam at once, and optionally to weld it all at once.

FIG. 16g is a three dimensional sketch of the general structure of such “clamps” 236, which may for example comprise wires in the shape of the cross section of the balloon at the level of the seam.

FIG. 16h is an end view of balloon 220′ during manufacturing using clamps 236, which are seen above and below seam 224, as they press flanges of both parts 228′ against each other, along all the seam line, and exit the balloon through neck 226.

Typically, at least two clamps 236 will be used for each seam, one above and one underneath each “flange” 230′. Optionally, each clamp 236 may comprise at least two parts, to facilitate insertion into the balloon and removal therefrom.

Welded Balloon with no Protruding Seam.

In some embodiments, such as examples shown in FIGS. 16i and 16j, balloon 240 with no protruding seam is manufactured. The seam may be created as an overlap 238 of the two balloon parts 228′ so that it does not protrude to either direction. Tools and methods similar to those described above for the inward facing seam balloon 220′ may be used for making balloon 240, with the difference that clamping of parts 228′ will be between a tool inside the balloon, and a tool outside the balloon.

Needle Electrodes.

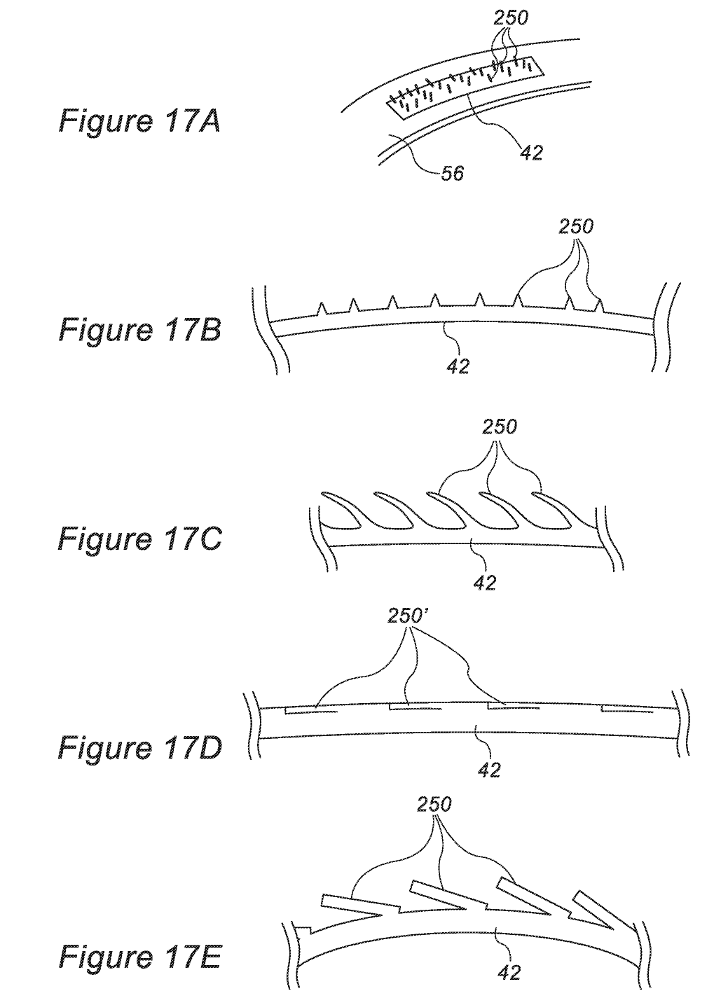

In some embodiments shown in FIGS. 17a-e, needle electrodes 250 protruding around the expandable element and entering into the tissue may be used.

FIG. 17a is a schematic three dimensional sketch of needle electrodes 250 protruding from electrode segment 42 over flexcircuit arm 56. This is merely by way of example, as needles 250 may extend from other electrode segments such as 41 or 43, or any other surface within ablation device 50 that may come into contact with the treated tissue.

Needles 250 may be manufactured as an integral part of flexible PCB 140. Alternatively they may be manufactured as separate elements that may be welded or otherwise connected to PCB 140. For example needles 250 may be made by laser cutting strips of nitinol.

In some embodiments, needles 250 may be fixed, i.e., protrude from the surface to a constant distance without change during the procedure. FIG. 17b is a schematic longitudinal section of segment 42 with such fixed needles 250.

Alternatively, needles 250 may change the degree of their protrusion from segment 42 (or any other surface) during the procedure. Typically in such embodiments, needles 250 would protrude less in the folded or compressed state of device 50, and protrude more during the deployed state.

In some embodiments, needles 250 may be self-deploying, i.e., they may possess the innate tendency to protrude radially from electrode segment 42. This may for example be achieved by heat treatment of the needle structure fixing the needles with an outward angle as shown in FIG. 17c which is a schematic longitudinal section along segment 42. In such embodiments, needles would be folded flat when pulled into outer sheath 78 in the folded or compressed state, and will spring open in the deployed state.

In other embodiments, needles 250 may be made of a shape memory alloy such as nitinol and may be heat treated in a way such as to assume a collapsed position when cold, and protrude radially when exposed to body temperature. Thus, needles 250 may be easily folded or compressed in the folded or compressed state of device 50, and may only protrude when inserted into the patient's body.

In yet other embodiments shown in FIGS. 17d-17e, needles 250 protrude only during expansion of expandable element 30.

As seen in FIG. 17d, which is a schematic longitudinal section along segment 42 in the folded or compressed state of device 50, needles 250′ are flat and parallel to segment 42 as long as segment 42 is flat (i.e., while in the folded or compressed state). As seen in FIG. 17e, which is a schematic longitudinal section along segment 42 in the expanded state of expandable element 30, in the expanded state of expandable element 30, PCB 140 may curve, segment 42 may assume a circular longitudinal section, and as a result needles 250 may protrude relative to the curved surface of segment 42.

The above are examples, other designs and methods known in the art may be used for causing needles 250 to be collapsed in the folded or compressed state of device 50 and assume a protruding position during deployed or expanded state.

The extent of protrusion of needles 250 may be predetermined and limited by design, depending on the treated organ and target tissue. For example, typically, for treatment in the urinary bladder, needles 250 may protrude between 0.1 mm to 1.5 mm from the surface of segment 42 (or any other surface). This distance is measured perpendicular to the surface, in other words, if at an angle as in FIG. 17e, the length of needles 250 may be longer than the above mentioned depth of protrusion into the tissue.

Typically, needles 250 may be used in combination with far field bipolar technology. However, needles 250 may also be used with other ablation technologies/modalities, for ablation in general and for TBP in particular.

Use of needles 250 in combination with the various above described methods of suction applied around the balloon may be of particular benefit.

The advantage of using needles 250 may be both in ensuring good tissue electrode-contact and in cases where it may be desirable to avoid ablation of superficial layers. In such cases, needles 250 may be insulated all over apart from at their tips.

Localized Treatment.

Although described above in the context of whole organ treatment, or treatment that targets a large area of an organ with a repetitive pattern (such as TBP for the bladder), in some embodiments, far field bipolar technology may be used for targeted treatment of localized, relatively small areas in an organ.

In some embodiments this may be achieved by the two poles of each set of electrodes having different surface areas, as mentioned above. For example, the pole at the target region may comprise electrodes with a small total surface area, while the electrodes at the other pole, where a lesion is not desired, may have a large total surface area. In such case, the large surface area pole serves a role similar to the “dispersive” electrode of monopolar ablation, despite being used here as bipolar.

The difference in surface areas between the “treating” and “dispersive” electrodes may typically be in a ratio of between 1:2 to 1:20, preferably between 1:5 to 1:10.

For the procedure that is the subject of this application, using “far-field bipolar” energy coupling, the “treating” and “dispersive” electrodes, which form the bipolar pair, are intended to have approximately the same surface area.

For example, in the bladder, the trigone may be specifically targeted. This may be done for example with the purpose of denervating the bladder. Alternatively this may be done for disrupting signal propagation within the bladder wall in the area of the trigone only, or for isolation of the trigone area.

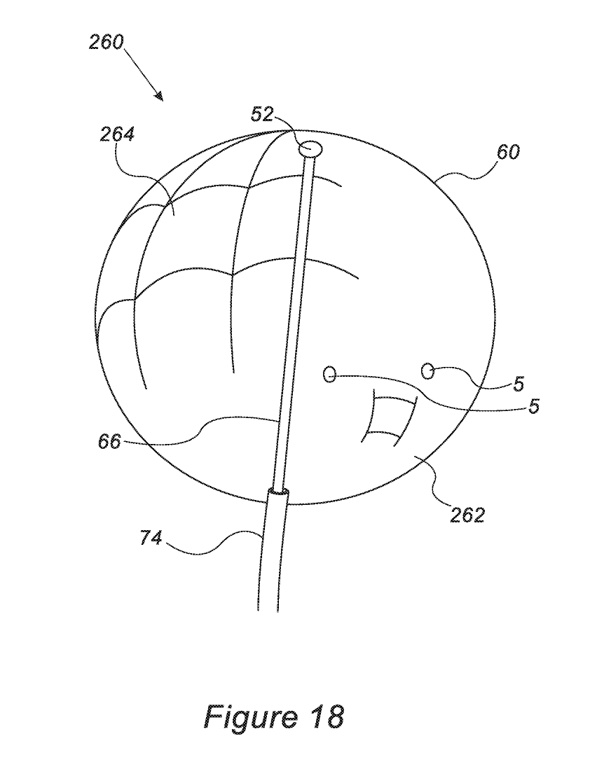

As an example, FIG. 18 is a schematic three dimensional sketch of localized treatment probe 260 which may be used for treating a urinary bladder trigone using far field bipolar. Probe 260 may be basically identical to probes 50 described above, with the main difference being in the position of the electrode segments. For the sake of clarity, only the electrode segments are shown, although typically they may be positioned on a flexible PCB very similar to PCB 140 described above.

As seen in FIG. 18, two short longitudinal electrode segments and two short circumferential electrodes segments (together treatment array 262) may be positioned on the lower hemisphere of balloon adjacent the proximal balloon neck, targeting the area of the trigone at a safe distance below the location of ureteral orifices 5. On the other side of balloon 60, opposite array 262, two long longitudinal electrodes segments and six long circumferential segments (together dispersive array 264) may be positioned. The electrode segments shown in FIG. 18 are merely for the purpose of illustration, and may be designed differently in shape, location, number, etc. The important point is that treatment array 262 may be much smaller in area than dispersive array 264. Thus, when delivering energy to electrodes in array 262 which may serve as one pole, while coupled with electrodes in array 264 which may serve as the opposite pole, a significant lesion may form at the treatment array 262, whereas no lesion, or a very superficial lesion may form at dispersive array 264.

In some embodiments, the bladder neck may be targeted, for example for inducing mechanical changes, for treating stress incontinence.

A localized area in the bladder dome may be treated for example for isolating that area following identification of an aberrant focus of activity there. Alternatively treatment for a localized area of the bladder using far field bipolar may be undertaken for treatment of a superficial tumor, bleeding, or any other bladder condition.

In some embodiments, the devices of the invention are further adapted to deliver therapeutic substances to the treated regions. This may for example be achieved by coating the treating electrodes with the drug that is to be delivered. Ablation of the tissue may increase its permeability and absorption of the drug. Release of the drug from the electrodes may be a result of the current flowing through the electrodes, a result of the increase in temperature, or both.

As an example, conditions that may be treated in this manner in the urinary bladder may include overactive bladder, bladder-detrusor dyssynergia, pelvic pain syndrome, hypoactive bladder, neoplastic disease.

Substances that may be delivered in this manner may for example include but are not limited to botulinum toxin, anticholinergics, and Glivec, to name a few.

Yet another embodiment may involve using a balloon which may be mirror like or opaque, except in specific lines where the ablation is desired, along which it may be at least partially transparent.

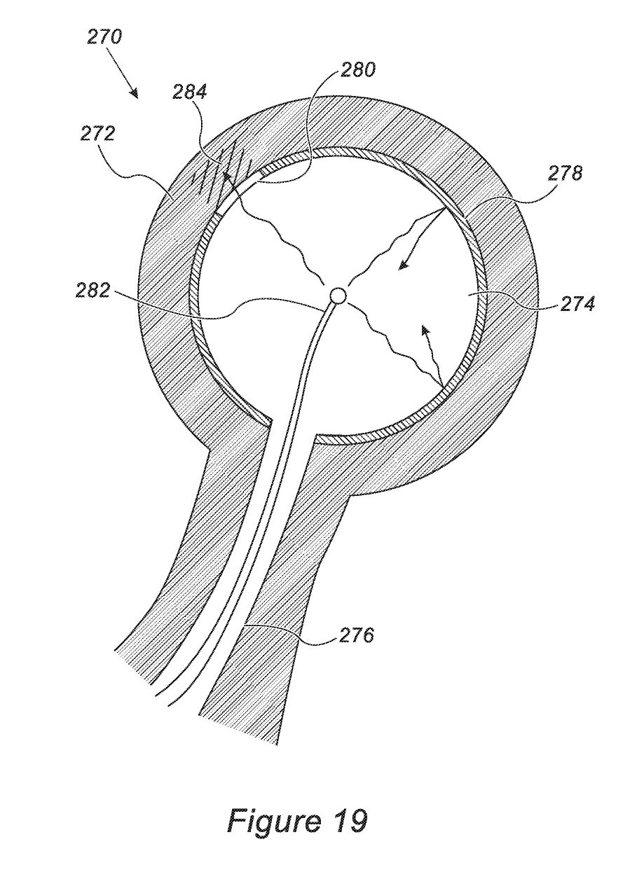

FIG. 19 is a schematic longitudinal cross section of such a mirror balloon device 270.

Shown in FIG. 19 is an organ 272, inside which is seen inflated balloon 274, which may have catheter 276 with at least one lumen for inflation.

Most of the surface area of balloon 274 may reflective or opaque to light coming from its interior (areas marked 278) while specific areas 280 of the balloon, may be transparent or semi-transparent. Areas 280 may have a linear shape or any other shape as necessary for creating the desired target ablation lesion.

The above may for example be achieved by application of a reflective or opaque coating to the interior or exterior of a transparent balloon, by manufacturing the balloon from reflective and transparent strips, or any other method.

A high intensity light source 282, for example an infrared light source, may be used to illuminate the inside of the balloon. Such light source may for example be an optic fiber transmitting laser light from an external source, or a small, yet powerful laser diode.

The light may illuminate the organ wall 272 only at the areas where the balloon is penetrable to light, thus producing ablation as determined by the transparent areas.

Optionally, different wavelengths can be used to target a specific layer or tissue type of the bladder wall.

Although described in the context of urinary bladder ablation for treatment of overactive bladder, it is clear that the devices and methods provided herein may be used in various other body organs and medical conditions.

These may include but are not limited to treating a urinary bladder for other micturition disorders such as detrusor-sphincter dyssynergia or pelvic pain syndrome, treating a uterus for irritable uterus or menorrhagia, treating a rectum, a large or a small bowel, for irritable bowel, treating a stomach for obesity, bronchi for asthma, a pulmonary artery or a cardiac atrium for atrial fibrillation, or a cardiac ventricle for ventricular tachycardia, uterus, pulmonary artery, cardiac atrium, cardiac ventricle, and the disorder is any of overactive bladder, obesity, asthma, atrial fibrillation, ventricular tachycardia.

Treatment of Underactive Bladder.

In some embodiments, the methods and devices described may be used to treat patients suffering from underactive bladder syndromes.

Detrusor underactivity, or underactive bladder (UAB), is defined as a contraction of reduced strength and/or duration resulting in prolonged bladder emptying and/or a failure to achieve complete bladder emptying within a normal time span. UAB can be observed in many neurologic conditions and myogenic failure. Diabetic cystopathy is the most important and inevitable disease developing from UAB, and can occur silently and early in the disease course. Careful neurologic and urodynamic examinations are necessary for the diagnosis of UAB. Proper management is focused on prevention of upper tract damage, avoidance of over distension, and reduction of residual urine. Scheduled voiding, double voiding, alpha blockers, and intermittent self-catheterization are the typical conservative treatment options. Sacral nerve stimulation may be an effective treatment option for UAB. New concepts such as stem cell therapy and neurotrophic gene therapy are being explored. Other new agents for UAB that act on prostaglandin E2 and EP2 receptors are currently under development.

For these embodiments, the device and methods described in the current invention may be adapted to achieve one or more of the desired effects: reduce the post voiding residual volume, decrease bladder compliance, induce (temporary) inflammation of the bladder wall, increase the afferent nerve signaling from the bladder, augment bladder contraction reflexes and/or awareness of bladder fullness, and/or cause partial denervation of the bladder.

Such treatment may be useful in treating patients who suffer from increased post voiding residual volume, and/or recurrent urinary tract infections, and/or difficulty with weaning from an indwelling urinary bladder catheter.

To achieve the desired effects and treat the listed clinical presentations, ablation device 50 may be adapted to create thermal effects in the bladder wall, with the intention of inducing various results, such as transient inflammation of the bladder wall. The treatment may be further adapted to temporarily damage the Urothelial layer only, enabling contact of urine with the underlying bladder wall layers—inducing inflammation and increased afferent nerve activity. Although the Urothelial layer is expected to quickly repair and return to normal, the underlying inflammation and the late effects of such inflammation (scarring, remodeling), are expected to be long lasting.

In some embodiments, only the dome of the bladder may be targeted. In some embodiments, the ablation may be applied to cause thermal damage through the entire bladder wall thickness, inducing inflammation of also the serosa covering the peritoneal parts of the bladder. In some embodiments, the ablation may be applied to damage the nerves supplying the bladder, effectively inducing a state of “neurogenic bladder” hyperactivity and increased bladder tone.

Generally, the ablation energy (duration times power) per bladder thickness for these indications may typically be 25% to 125% greater than the ablation energy applied to treat overactive bladder syndromes. When the bladder wall of an underactive bladder patient is thinner than the bladder wall of the average overactive bladder patient, the same power settings may be used, effectively achieving higher power settings per bladder thickness.

In some embodiments, the treatment of underactive bladder may be achieved by the eventual scarring and contraction of the ablation areas. For these embodiments, symmetric and continuous ablation lines may be preferred. The use of symmetric ablation patterns may allow uniform contraction of the bladder.

In some embodiments, the ablation may be applied with the bladder filled to a volume of 150 cc to 250 cc, and the bladder may subsequently be allowed to heal for two weeks or more, while the bladder may be kept at lower volumes (completely empty using an indwelling urinary catheter, or by frequent intermittent catheterizations and/or scheduled urinations). Using this method, the healing (with associated scarring and remodeling) of the ablations may somewhat affix the bladder at the empty state, reducing the volume of the bladder and bladder compliance, effectively increasing the micturition activities of the bladder.

While the exact mechanisms involved in causing bladder underactivity are still under much debate, most researchers agree that bladders that exhibit underactivity actually exhibit increased responsiveness to electrical field stimulation. (Yoshimura N. -Recent advances in understanding the biology of diabetes associated bladder complications. BJU Int. 2005 April; 95(6):733-8). These findings may suggest that bladder underactivity is caused or mediated, at least in part, by propagation of electrical fields within the bladder wall. Thus, in some embodiments of the current invention, bladder partitioning may be applied to treat underactive bladder syndromes. Partitioning of the bladder may be applied to block (limit) dissemination of relaxation signals throughout the bladder. As opposed to the common belief that bladder underactivity is caused by a lack of electrical and/or mechanical activity, the inventors believe that the pan-bladder underactivity may be caused and/or aggravated by electrical signaling through the bladder wall (much as in bladder over-activity). Thus, bladder partitioning to limit the conduction of electrical signals in the bladder as a whole is anticipated to alleviate the exaggerated bladder relaxation at the root of bladder underactivity.

In some embodiments, the partitioning of the bladder may be used to isolate sections of the bladder from efferent nerve activity (denervation). While complete denervation of the bladder may be substantially impossible from within the bladder (unless causing excessive damage to the bladder wall and surrounding tissues), isolation of bladder segments by creation of ablation lines around the entire periphery of a bladder zone may maximize the chances of complete denervation of at least certain zones of the bladder. It is believed that achieving substantially complete denervation of even a few bladder zones may induce increased tone and activity in such zones, effectively alleviating the bladder underactivity.

In some embodiments of the current invention, the pattern of ablation used to treat bladder underactivity may comprise a circumferential ablation line approximately at mid bladder height, with eight splines crossing the bladder dome (much like a sliced pizza would look like from above). This pattern may ensure the ureteral orifices and the trigone are spared, and may target the bladder dome which is most prone to over stretching and flaccidity (the lower parts of the bladder are limited by the pelvic organs).

A larger total ablation surface area may be expected to result with a greater reduction in post voiding residual volume, and may be more effective in treating underactive bladder. Thus, alternative patterns of bladder partitioning having a larger total ablation surface area may be preferable for treating underactive bladder. These patterns may include, but are not limited to, a pattern similar to that described above, having a greater number of longitudinal lines e.g., sixteen instead of eight, and/or a greater number of circumferential lines e.g. two instead of one.

In some embodiments of the current invention, the devices and techniques described may be used in conjunction with chemical agents that may be applied to the bladder following the ablations. In these embodiments, the ablation serves to remove the urothelial barrier over ablation lines, thus effectively targeting and facilitating the passage of chemical agents to the specific bladder wall areas beneath the ablations. In some embodiments, the chemical agent is botulinum toxin, and the end effect is reduced bladder activity (treating overactive bladder). In some embodiments, the chemical agent is Talc or a similar agent known to induce fibrosis, and the end effect is reduction in bladder volume and increase in bladder activity (treating underactive bladder). In some embodiments, the chemical agent is an inflammation inducing agent (biological foreign material or other irritative materials), to induce scarring. In some embodiments, the chemical agent applied is an acid or base, effectively thinning the bladder wall at the targeted (exposed) areas, to encourage the development of bladder diverticuli, to increase bladder compliance to treat overactive bladder.

In some embodiments of the current invention, the pattern of ablation described above (circumferential line and eight longitudinal splines) may be used to affix the bladder dome to the peritoneal cavity floor, effectively “capping” the bladder dome with thickened contracted tissue. This technique may be used to treat cystocele and/or to reduce symptoms of stress incontinence and/or reduce reflux of urine back to the ureters. The same effect may also be useful in the treatment of underactive bladders, by limiting the maximal volume of the bladder and increasing bladder tone (reducing bladder compliance).

In some embodiments, a mesh placed over the peritoneal part of the bladder may be used to achieve the same effect, using ablation, or optionally without using ablation at all. The mesh may be completely flat, or somewhat dome shaped to allow some flexibility of the bladder dome.

TABLE 1
Reference Number Chart

| Label | Component name |
|---|---|
| 1 | Urinary bladder |
| 2 | Bladder wall |
| 3 | Bladder lumen |
| 4 | Bladder outlet |
| 5 | Ureteral orifice |
| 6 | Urothelium |
| 7 | Detrusor |
| 8 | Adventitia |
| 9 | Bladder apex |
| 10 | Ablation [pattern |
| 11 | Circumferential line |
| 12 | Longitudinal spline |
| 30 | Spherical expandable element |
| 40 | Electrode structure |
| 41 | Distal longitudinal electrode segments |
| 42 | Proximal longitudinal electrode segments |
| 43 | Circumferential electrode segments |
| 45 | Electrode structure center |
| 50 | Ablation Device |
| 52 | Atraumatic cap |
| 54 | flexcircuit plate |
| 56 | flexcircuit arms |
| 58 | Tip plug |
| 60 | balloon |
| 62 | distal balloon neck |
| 64 | proximal balloon neck |
| 66 | inner shaft |
| 68 | distal ring |
| 70 | stopper |
| 72 | Proximal ring |
| 74 | Outer shaft |
| 76 | flexcircuit arms proximal ring |
| 78 | outer sheath |
| 79 | Outer sheath distal tip |
| 80 | sliding valve |
| 82 | sheath port |
| 84 | Valve seal |
| 86 | sliding stopper |
| 90 | handle |
| 92 | housing |
| 94 | slot |
| 96 | outer shaft base |
| 98 | outer shaft seal |
| 100 | retraction knob |
| 102 | inner shaft base |
| 104 | wires |
| 106 | Electric plug |
| 108 | Inflation tube |
| 110 | stopcock |
| 120 | Locking mechanism |
| 122 | lever |
| 124 | tooth |
| 126 | Release button |
| 128 | hinge |
| 130 | Proximal inner shaft openings |
| 132 | Distal inner shaft openings |
| 140 | Flexible PCB |
| 142 | Flexcircuit arms proximal ends |
| 144 | Distal connectors |
| 146 | proximal connectors |
| 147 | Insulated tracks |
| 150 | Flexcircuit hinge |
| 152 | Tightening wire |
| 154 | loop |
| 156 | Middle hinge |
| 160 | Upper circumferential electrode segments |
| 162 | Lower circumferential electrode |
| | segments |
| 164 | Anterior longitudinal electrode segments |
| 166 | Posterior longitudinal electrode segments |
| 168 | Diagonal electrode segments |
| 200 | Sheath suction holes |
| 201 | Distal suction lumen port |
| 202 | Distal suction lumen |
| 203 | Distal suction openings |
| 204 | Miniature fluid channels |
| 206 | Balloon channel |
| 210 | Gaps |
| 212 | gelatinous material layer |
| 220 | Welded balloon |
| 222 | Outwards facing balloon seam |
| 224 | Inward facing seam |
| 226 | Balloon neck |
| 228 | Balloon part |
| 230 | Balloon part flange |
| 232 | forceps |
| 234 | Rollers |
| 236 | Clamps |
| 238 | overlap |
| 240 | Balloon with no protruding seam |
| 250 | Needle electrodes |
| 260 | Localized treatment probe |
| 262 | Treatment array |
| 264 | Dispersive array |
| 270 | Mirror balloon devie |
| 272 | Organ |
| 274 | Mirror balloon |
| 276 | catheter |
| 278 | Mirror/opaque areas |
| 280 | Transparent areas |

While preferred embodiments of the current disclosure have been shown and described herein, it will be obvious to those skilled in the art that such embodiments are provided by way of example only. Numerous variations, changes, and substitutions will now occur to those skilled in the art without departing from the scope of the present disclosure. It should be understood that various alternatives to the embodiments described herein may be employed. It is intended that the following claims define the scope of the invention and that methods and structures within the scope of these claims and their equivalents be covered thereby.

1. A device for treating a disorder in a hollow body organ, said device comprising:
a shaft having a distal tip;
at least one set of bipolar electrodes; and
an expandable member configured to radially expand the at least one set of bipolar electrodes from a folded or compressed position to a deployed position,
wherein each set of bipolar electrodes comprises at least one first polarity electrode and at least one second polarity electrode,
wherein a total tissue contact area of the at least one first polarity electrode of each set of bipolar electrodes is substantially equal to a total surface area of the at least one second polarity electrode of the same set of bipolar electrodes,
wherein the device is configured for creating a predetermined pattern of electrically isolated tissue regions having reduced electrical propagation in an inner wall of the hollow body organ such that electrical propagation through the hollow body organ as a whole is reduced, and
wherein each electrode comprises an elongate conductor.

2.-4. (canceled)

5. The device of claim 1, wherein the at least one set of bipolar electrodes comprises at least one set of longitudinal electrodes which are configured to be substantially parallel to the longitudinal axis of the shaft in the deployed position and at least one set of circumferential electrodes which are configured to be substantially transverse to the longitudinal axis of the shaft in the deployed position.

6. The device of claim 5, wherein the at least one set of longitudinal electrodes are structured as eight splines from the device tip to the equator of the expandable element, and the at least one set of circumferential electrodes is structured as one line around the equator of the expandable element.

7.-9. (canceled)

10. The device of claim 5, wherein the longitudinal electrodes comprise a flexible printed circuit material, and the circumferential electrodes comprise a wire or braid.

11. The device of claim 1, wherein at least some of the electrodes comprise a flexible printed circuit material.

12. The device of claim 1, wherein all first polarity electrode segments of each set and all second polarity electrode segments of each set are connected to each other but not to any other electrode segments via a printed circuit board located at the distal tip of the shaft.

13. The device of claim 1, wherein the tissue contact area of each electrode set is between 1 mm² and 50 mm².

14. The device of claim 1, further comprising one or more wires configured to deliver power to a PCB pass via the shaft.

15. The device of claim 1, further comprising an atraumatic cap at the distal tip of the shaft.

16. The device of claim 1, wherein the expandable member comprises a balloon or bladder made of a non-compliant material.

17. (canceled)

18. The device of claim 1, wherein the at least one set of bipolar electrodes is printed on the expandable member.

19. (canceled)

20. The device of claim 1, wherein the expandable member is made of a compliant material.

21. The device of claim 1, wherein the electrodes create a pattern that is asymmetrical.

22. The device of claim 1, wherein the pattern is configured to spare an area of the hollow organ.

23.-24. (canceled)

25. The device of claim 1, wherein the hollow organ is any of a urinary bladder, uterus, rectum, large or small bowel, stomach, pulmonary artery, cardiac atrium, cardiac ventricle, and the disorder is any of overactive bladder, Detrusor-sphincter dyssynergia, irritable uterus, menorrhagia, irritable bowel, obesity, asthma, atrial fibrillation, ventricular tachycardia.

26. The device of claim 1, wherein the at least one set of bipolar electrodes comprises a conductive flexible or gelatinous material layer on surfaces thereof.

27.-30. (canceled)

31. A device for treating a disorder in a hollow body organ, the device comprising:
a handle having a distal end, a proximal end, and a slot;
an inner shaft having a distal tip, a proximal end, a stopper and at least one opening;
an outer shaft slideably positioned over the inner shaft and having a distal tip, a proximal end, a seal, and an outer shaft base;
an outer sheath slideably positioned over the outer shaft and having a distal end, a proximal end, and a valve;
at least one set of electrodes each having a distal end and a proximal end and comprising at least one electrode segment; and
a balloon having a distal leg and a proximal leg;
wherein the proximal end of the inner shaft is connected to the handle,
wherein the outer shaft base further comprises a retraction knob which slideably protrudes through the slot of the handle,
wherein an inflation tube and wires enter the handle and are sealed to the inner shaft,
wherein the distal leg of the balloon is connected to the inner shaft proximate the distal tip of the inner shaft and the proximal leg of the balloon is connected to the outer shaft proximal the distal tip of the outer shaft,
wherein the wires pass through the inner shaft and out of the distal tip of the shaft and connect to the at least one set of electrodes, and
wherein the proximal end of the at least one electrodes is connected as a ring slideably positioned over the outer shaft proximal to the proximal leg of the balloon.

32. The device of claim 31, wherein the device has a folded or compressed position and a deployed position and further comprises an atraumatic cap connected to the inner shaft distal tip,
wherein the atraumatic cap is configured to either partially or completely cover the outer sheath distal end when in the folded or compressed position,
wherein the outer sheath is configured to expose the electrodes when pulled proximally,
wherein the balloon is configured to radially expand the electrodes when inflated,
wherein the electrodes are configured to deliver energy to the hollow organ, and
wherein the outer shaft is configured to stretch the balloon and collapse the electrodes when pulled proximally by the retraction knob.

33. The device of claim 31, wherein the at least one set of electrodes comprises longitudinal and circumferential electrodes.

34. The device of claim 33, wherein the longitudinal electrodes comprise a flexible printed circuit material.

35. The device of claim 33, wherein the circumferential electrodes comprise a flexible printed circuit material.

36. The device of claim 33, wherein the circumferential electrodes are foldable.

37.-41. (canceled)

42. The device of claim 31, wherein the electrodes create a pattern that is asymmetrical.

43. The device of claim 42, wherein the pattern is configured to spare an area of the hollow organ.

44. (canceled)

45. The device of claim 31, wherein the balloon is made of a compliant material.

46. The device of claim 31, wherein the balloon is made of a non-compliant material.

47. The device of claim 31, wherein the hollow organ is any of a urinary bladder, uterus, rectum, large or small bowel, stomach, pulmonary artery, cardiac atrium, cardiac ventricle, and the disorder is any of overactive bladder, Detrusor-sphincter dyssynergia, irritable uterus, menorrhagia, irritable bowel, obesity, asthma, atrial fibrillation, ventricular tachycardia.

48. The device of claim 31, wherein the at least one set of bipolar electrodes comprises a conductive flexible or gelatinous material layer on surfaces thereof.

49.-70. (canceled)
